(19) United States
(12) Patent Application Publication
(10) Pub. No.: US 2002/0068054 A1
(43) Pub. Date: Jun. 6, 2002

(54) Therapeutic and cosmetic uses of heparanases

(75) Inventors: Neta Ilan, Rehovot (IL); Israel Vlodavsky, Mevaseret Zion (IL); Oron Yacoby-Zeevi, Meitar (IL); Iris Pecker, Rishon Lezion (IL)

Correspondence Address: G. E. EHRLICH (1995) LTD., c/o ANTHONY CASTORINA, SUITE 207, 2001 JEFFERSON DAVIS HIGHWAY, ARLINGTON, VA, 22202, US

(73) Assignee: Insight Strategy & Marketing Ltd. and Hadasit Medical Research Services and Development Ltd.

(21) Appl. No.: 09/727,479

(22) Filed: Dec. 4, 2000

Related U.S. Application Data
(60) Provisional application No. 60/231,551, filed on Sep. 11, 2000.
(60) Provisional application No. 60/244,593, filed on Nov. 1, 2000.

(51) Int. Cl. A61K 38/47

(52) U.S. Cl. 424/94.61

(57) ABSTRACT

Methods and compositions for inducing and/or accelerating wound healing and/or angiogenesis via the catalytic activity of heparanase are disclosed.

This is a continuation-in-part of U.S. Provisional Patent Application Nos. 60/231,551, filed Sep. 11, 2000, and 60/244,593, filed Nov. 1, 2000.

### FIELD AND BACKGROUND OF THE INVENTION

The present invention relates to therapeutic and cosmetic uses of heparanase. More particularly, the present invention relates to the use of heparanase for induction and/or acceleration of wound healing and/or angiogenesis and for cosmetic applications, including skin and hair treatment and conditioning.

Proteoglycans (PGs)

Proteoglycans (previously named mucopolysaccharides) are remarkably complex molecules and are found in every tissue of the body. They are associated with each other and also with other major structural components, such as collagen and elastin. Some PGs interact with certain adhesive proteins, such as fibronectin and laminin.

Glycosaminoglycans (GAGs)

Glycosaminoglycans (GAGs) proteoglycans are polyanions and hence bind polycations and cations, such as Na⁺ and K⁺. This latter ability attracts water by osmotic pressure into the extracellular matrix and contributes to its turgor. GAGs also gel at relatively low concentrations. The long extended nature of the polysaccharide chains of GAGs and their ability to gel, allow relatively free diffusion of small molecules, but restrict the passage of large macromolecules. Because of their extended structures and the huge macromolecular aggregates they often form, they occupy a large volume of the extracellular matrix relative to proteins [Murry R K and Keeley F W; Harper's Biochemistry, 24th Ed. Ch. 57. pp. 667-85].

Heparan Sulfate (HS) Proteoglycans

Heparan sulfate (HS) proteoglycans are acidic polysaccharide-protein conjugates associated with cell membranes and extracellular matrices. They bind avidly to a variety of biologic effector molecules, including extracellular matrix components, growth factor, growth factor binding proteins, cytokines, cell adhesion molecules, proteins of lipid metabolism, degradative enzymes, and protease inhibitors. Owing to these interactions, heparan sulfate proteoglycans play a dynamic role in biology, in fact most functions of the proteoglycans are attributable to the heparan sulfate chains, contributing to cell-cell interactions and cell growth and differentiation in a number of systems. It maintains tissue integrity and endothelial cell function. It serves as an adhesion molecule and presents adhesion-inducing cytokines (especially chemokines), facilitating localization and activation of leukocytes. The adhesive effect of heparan sulfate-bound chemokines can be abrogated by exposing the extracellular matrices to heparanase before or after the addition of chemokines. Heparan sulfate modulates the activation and the action of enzymes secreted by inflammatory cells. The function of heparan sulfate changes during the course of the immune response are due to changes in the metabolism of heparan sulfate and to the differential expression of and competition between heparan sulfate-binding molecules [Selvan R S et al.; Ann. NY Acad. Sci. 1996; 797:127-139]

Other PGs and GAGs, such as hyaluronic acid, chondroitin sulfates, keratan sulfates I, II, dermatan sulfate and heparin have also important physiological functions.

GAG Degrading Enzymes

Degradation of GAGs is carried out by a battery of lysosomal hydrolases. These include certain endoglycosidases, such as, but not limited to, mammal heparanase (U.S. Pat. No. 5,968,822 for recombinant and WO91/02977 for native human heparanase) and connective tissue activating peptide III (CTAP, WO95/04158 for native and U.S. Pat. No. 4,897,348 for recombinant CTAP) which degrade heparan sulfate and to a lesser extent heparin; heparinase I, II and III (U.S. Pat No. 5,389,539 for the native form and WO95/34635 A1, U.S. Pat. No. 5,714,376 and U.S. Pat. No. 5,681,733 for the recombinant form), e.g., from Flavobacterium heparinum and Bacillus sp., which cleave heparin-like molecules; heparitinase T-I, T-II, T-III and T-VI from Bacillus circulars (U.S. Pat. No. 5,405,759, JO 4278087 and JP04-278087); β-glucuronidase; chondroitinase ABC (EC 4.2.2.4) from Proteus vulgaris, AC (EC 4.2.2.5) from Arthrobacter aurescens or Flavobacterium heparinum, B and C (EC 4.2.2) from Flavobacterium heparinum which degrade chondroitin sulfate; hyaluronidase from sheep or bovine testes which degrade hyaluronidase and chondroitin sulfate; various exoglycosidases (e.g., β-glucuronidase EC 3.2.1.31) from bovine liver, mollusks and various bacteria; and sulfatases (e.g., iduronate sulfatase) EC 3.1.6.1 from limpets (Patella vulgaris), Aerobacter aerogens, Abalone entrails and Helix pomatia, generally acting in sequence to degrade the various GAGs.

Heparanase

One important enzyme involved in the catabolism of certain GAGs is heparanase. It is an endo-β-glucuronidase that cleaves heparan sulfate at specific interchain sites. Interaction of T and B lymphocytes, platelets, granulocytes, macrophages and mast cells with the subendothelial extracellular matrix (ECM) is associated with degradation of heparan sulfate by heparanase activity. The enzyme is released from intracellular compartments (e.g., lysosomes or specific granules) in response to various activation signals (e.g., thrombin, calcium ionophore, immune complexes, antigens and mitogens), suggesting its regulated involvement in inflammation and cellular immunity [Vlodavsky I et al.; Invasion Metas. 1992; 12(2):112-27].

Cloning and Expression of the Heparanase Gene

A purified fraction of heparanase isolated from human hepatoma cells was subjected to tryptic digestion. Peptides were separated by high pressure liquid chromatography and micro sequenced. The sequence of one of the peptides was used to screen data bases for homology to the corresponding back translated DNA sequence. This procedure led to the identification of a clone containing an insert of 1020 base pairs (bp) which included an open reading frame of 963 bp followed by 27 bp of 3′ untranslated region and a poly A tail. The new gene was designated hpa. Cloning of the missing 5′ end of hpa was performed by PCR amplification of DNA from placenta cDNA composite. The entire heparanase cDNA was designated phpa. The joined cDNA fragment contained an open reading frame which encodes a polypeptide of 543 amino acids with a calculated lo molecular weight of 61,192 daltons. Cloning an extended 5′ sequence was enabled from the human SK-hep I cell line by PCR amplification using the Marathon RACE system. The 5′ extended sequence of the SK-hep1 hpa cDNA was assembled with the sequence of the hpa cDNA isolated from human placenta. The assembled sequence contained an open reading frame 15 which encodes a polypeptide of 592 amino acids with a calculated molecular weight of 66,407 daltons. The cloning procedures are described in length in U.S. Pat. No. 5,968,822; U.S. patent application Ser. Nos. 09/109,386, and 09/258,892; and PCT Application No. US98/17954.

The ability of the hpa gene product to catalyze degradation of heparan sulfate (HS) in vitro was examined by expressing the entire open reading frame of hpa in High five and Sf21 insect cells, and the mammalian human 293 embryonic kidney cell line expression systems. Extracts of infected cells were assayed for heparanase catalytic activity. For this purpose, cell lysates were incubated with sulfate labeled, ECM-derived HSPG (peak I), followed by gel filtration analysis (Sepharose 6B) of the reaction mixture. While the substrate alone consisted of high molecular weight material, incubation of the HSPG substrate with lysates of cells infected with hpa containing virus resulted in a complete conversion of the high molecular weight substrate into low molecular weight labeled heparan sulfate degradation fragments (see, for example, U.S. patent application Ser. No. 09/260,038).

In subsequent experiments, the labeled HSPG substrate was incubated with the culture medium of infected High Five and Sf21 cells. Heparanase catalytic activity, reflected by the conversion of the high molecular weight HSPG substrate into low molecular weight HS degradation fragments, was found in the culture medium of cells infected with the pFhpa virus, but not the control pF1 virus.

Altogether, these results indicate that the heparanase enzyme is expressed in an active form by cells infected with Baculovirus or mammalian expression vectors containing the newly identified human hpa gene.

In other experiments, it was demonstrated that the heparanase enzyme expressed by cells infected with the pFhpa virus is capable of degrading HS complexed to other macromolecular constituents (e.g., fibronectin, laminin, collagen) present in a naturally produced intact ECM (09/260,038), in a manner similar to that reported for highly metastatic tumor cells or activated cells of the immune system [Vlodavsky, I., Eldor, A., Haimovitz-Friedman, A., Matzner, Y., Ishai-Michaeli, R., Levi, E., Bashkin, P., Lider, O., Naparstek, Y., Cohen, I. R., and Fuks, Z. (1992) Expression of heparanase by platelets and circulating cells of the immune system: Possible involvement in diapedesis and extravasation. Invasion & Metastasis, 12, 112-127; Vlodavsky, I., Mohsen, M., Lider, O., Ishai-Michaeli, R., Ekre, H.-P., Svahn, C. M., Vigoda, M., and Peretz, T. (1995). Inhibition of tumor metastasis by heparanase inhibiting species of heparin. Invasion & Metastasis, 14: 290-302].

Latent and Active Forms of the Heparanase Protein

The apparent molecular size of the recombinant enzyme produced in the baculovirus expression system was about 65 kDa. This heparanase polypeptide contains 6 potential N-glycosylation sites. Following deglycosylation by treatment with peptide N-glycosidase, the protein appeared as a 57 kDa band. This molecular weight corresponds to the deduced molecular mass (61,192 daltons) of the 543 amino acid polypeptide encoded by the full length hpa cDNA after cleavage of the predicted 3 kDa signal peptide. No further reduction in the apparent size of the N-deglycosylated protein was observed following concurrent O-glycosidase and neuraminidase treatment. Deglycosylation had no detectable effect on enzymatic activity.

Expression of the full length heparanase polypeptide in mammalian cells (e.g., 293 kidney cells, CHO) yielded a major protein of about 50 kDa and a minor of about 65 kDa in cell lysates. Comparison of the enzymatic activity of the two forms, revealed that the 50 kDa enzyme is at least 100-200 fold more active than the 65 kDa form. A similar difference was observed when the specific activity of the recombinant 65 kDa enzyme was compared to that of the 50 kDa heparanase preparations purified from human platelets, SK-hep-1 cells, or placenta. These results suggest that the 50 kDa protein is a mature processed form of a latent heparanase precursor. Amino terminal sequencing of the platelet heparanase indicated that cleavage occurs between amino acids Gln¹⁵⁷ and Lys¹⁵⁸. As indicated by the hydropathic plot of heparanase, this site is located within a hydrophillic peak, which is likely to be exposed and hence accessible to proteases.

According to Fairbank et al. (57) the precursor is cleaved at three sites to form a heterodimer of a 50 kDa polypeptide (the mature form) that is associated with a 8 kDa peptide.

Purification of the Recombinant Heparanase Enzyme

Sf21 insect cells were infected with pFhpa virus and the culture medium was applied onto a heparin-Sepharose column. Fractions were eluted with a salt gradient (0.35-2.0 M NaCl) and tested for heparanase catalytic activity and protein profile (SDS/PAGE followed by silver staining). Heparanase catalytic activity correlated with the appearance of a about 63 kDa protein band in fractions 19-24, consistent with the expected molecular weight of the hpa gene product. Active fractions eluted from heparin-Sepharose were pooled, concentrated and applied onto a Superdex 75 FPLC gel filtration column. Aliquots of each fraction were tested for heparanase catalytic activity and protein profile. A correlation was found between the appearance of a major protein (approximate molecular weight of 63 kDa) in fractions 4-7 and heparanase catalytic activity. This protein was not present in medium conditioned by control non-infected Sf21 cells subjected to the same purification protocol. Recently, an additional purification protocol was applied, using a single step chromatography with source-S ion exchange column.

Using this protocol P65 heparanase is purified from conditioned medium of CHO clones overexpressing and secreting recombinant human heparanase precursor, while the processed P50 heparanase is purified from cell extracts of similar CHO clones which overexpress and accumulate mature P50 heparanase. This purification resulted in a protein purified to a degree of 90%. Further details concerning heparanase production and purification procedures are disclosed in U.S. patent application Ser. No. 15 09/071,618, which is incorporated by reference as if fully set forth herein.

Recombinantly modified heparanases are also known. To this end, see U.S. patent application Ser. No. 09/260,038.

Involvement of Heparanase in Tumor Cell Invasion and Metastasis

Circulating tumor cells arrested in the capillary beds of different organs must invade the endothelial cell lining and degrade its underlying basement membrane (BM) in order to escape into the extravascular tissue(s) where they establish metastasis [Liotta, L. A., Rao, C. N., and Barsky, S. H. (1983). Tumor invasion and the extracellular matrix. Lab. Invest., 49, 639-649]. Several cellular enzymes (e.g., collagenase IV, plasminogen activator, cathepsin B, elastase) are thought to be involved in degradation of the BM [Liotta, L. A., Rao, C. N., and Barsky, S. H. (1983). Tumor invasion and the extracellular matrix. Lab. Invest., 49, 639-649]. Among these enzymes is an endo-β-D-glucuronidase (heparanase) that cleaves HS at specific intrachain sites [Vlodavsky, I., Eldor, A., Haimovitz-Friedman, A., Matzner, Y., Ishai-Michaeli, R., Levi, E., Bashkin, P., Lider, O., Naparstek, Y., Cohen, I. R., and Fuks, Z. (1992). Expression of heparanase by platelets and circulating cells of the immune system: Possible involvement in diapedesis and extravasation. Invasion & Metastasis, 12, 112-127; Nakajima, M., Irimura, T., and Nicolson, G. L. (1988). Heparanase and tumor metastasis. J. Cell. Biochem., 36, 157-167; Vlodavsky, I., Fuks, Z., Bar-Ner, M., Ariav, Y., and Schirrmacher, V. (1983). Lymphoma cell mediated degradation of sulfated proteoglycans in the subendothelial extracellular matrix: Relationship to tumor cell metastasis. Cancer Res., 43, 2704-2711; Vlodavsky, I., Ishai-Michaeli, R., Bar-Ner, M., Fridman, R., Horowitz, A. T., Fuks, Z. and Biran, S. Involvement of heparanase in tumor metastasis and angiogenesis. Is. J. Med. 24:464-470, 1988]. HS degrading heparanase activity was found to correlate with the metastatic potential at mouse lymphoma cells [Vlodavsky, I., Fuks, Z., Bar-Ner, M., Ariav, Y., and Schirrmacher, V. (1983). Lymphoma cell mediated degradation of sulfated proteoglycans in the subendothelial extracellular matrix: Relationship to tumor cell metastasis. Cancer Res., 43, 2704-2711], fibrosarcoma and melanoma [Nakajima, M., Irimura, T., and Nicolson, G. L. (1988). Heparanase and tumor metastasis. J. Cell. Biochem., 36, 157-167]. The same is true for human breast, bladder and prostate carcinoma cells [see U.S. patent application Ser. No. 09/109,386, which is incorporated by reference as if fully set forth herein]. Moreover, elevated levels of heparanase were detected in sera [Nakajima, M., Irimura, T., and Nicolson, G. L. (1988). Heparanase and tumor metastasis. J. Cell. Biochem., 36, 157-167] and urine (U.S. patent application Ser. No. 09/109,386) of metastatic tumor bearing animals and cancer patients and in tumor biopsies [Vlodavsky, I., Ishai-Michaeli, R., Bar-Ner, M., Fridman, R., Horowitz, A. T., Fuks,Z. and Biran, S. Involvement of heparanase in tumor metastasis and angiogenesis. Is. J. Med. 24:464-470, 1988]. Treatment of experimental animals with heparanase alternative substrates and inhibitor (e.g., non-anticoagulant species of low molecular weight heparin, laminarin sulfate) markedly reduced (>90%) the incidence of lung metastases induced by B16 melanoma, Lewis lung carcinoma and mammary adenocarcinoma cells [Vlodavsky, I., Mohsen, M., Lider, O., Ishai-Michaeli, R., Ekre, H.-P., Svahn, C. M., Vigoda, M., and Peretz, T. (1995). Inhibition of tumor metastasis by heparanase inhibiting species of heparin. Invasion & Metastasis, 14: 290-302; Nakajima, M., Irimura, T., and Nicolson, G. L. (1988). Heparanase and tumor metastasis. J. Cell. Biochem., 36, 157-167; Parish, C. R., Coombe, D. R., Jakobsen, K. B., and Underwood, P. A. (1987). Evidence that sulfated polysaccharides inhibit tumor metastasis by blocking tumor cell-derived heparanase. Int. J. Cancer, 40, 511-517], indicating that heparanase inhibitors may be applied to inhibit tumor cell invasion and metastasis.

The studies on the control of tumor progression by its local environment, focus on the interaction of cells with the extracellular matrix (ECM) produced by cultured corneal and vascular endothelial cells (EC) [Vlodavsky, I., Liu, G. M., and Gospodarowicz, D. (1980). Morphological appearance, growth behavior and migratory activity of human tumor cells maintained on extracellular matrix vs. plastic. Cell, 19, 607-616; Vlodavsky, I., Bar-Shavit, R., Ishai-Michaeli, R., Bashkin, P., and Fuks, Z. (1991). Extracellular sequestration and release of fibroblast growth factor: a regulatory mechanism? Trends Biochem. Sci., 16, 268-271]. This ECM closely resembles the subendothelium in vivo in its morphological appearance and molecular composition. It contains collagens (mostly type III and IV, with smaller amounts of types I and V), proteoglycans (mostly heparan sulfate- and dermatan sulfate-proteoglycans, with smaller amounts of chondroitin sulfate proteoglycans), laminin, fibronectin, entactin and elastin [Parish, C. R., Coombe, D. R., Jakobsen, K. B., and Underwood, P. A. (1987). Evidence that sulfated polysaccharides inhibit tumor metastasis by is blocking tumor cell-derived heparanase. Int. J. Cancer, 40, 511-517; Vlodavsky, I., Liu, G. M., and Gospodarowicz, D. (1980). Morphological appearance, growth behavior and migratory activity of human tumor cells maintained on extracellular matrix vs. plastic. Cell, 19, 607-616]. The ability of cells to degrade HS in the ECM was studied by allowing cells to interact with a metabolically sulfate labeled ECM, followed by gel filtration (Sepharose 6B) analysis of degradation products released into the culture medium [Vlodavsky, I., Fuks, Z., Bar-Ner, M., Ariav, Y., and Schirrmacher, V. (1983). Lymphoma cell mediated degradation of sulfated proteoglycans in the subendothelial extracellular matrix: Relationship to tumor cell metastasis. Cancer Res., 43, 2704-2711]. While intact HSPG are eluted next to the void volume of the column (Kav<0.2, Mr of about 0.5×10⁶), labeled degradation fragments of HS side chains are eluted more toward the Vt of the column (0.5<kav<0.8, Mr of about 5-7×10³) [Vlodavsky, I., Fuks, Z., Bar-Ner, M., Ariav, Y., and Schirrmacher, V. (1983). Lymphoma cell mediated degradation of sulfated proteoglycans in the subendothelial extracellular matrix: Relationship to tumor cell metastasis. Cancer Res., 43, 2704-2711]. Compounds which efficiently inhibit the ability of heparanase to degrade the above-described naturally produced basement membrane-like substrate, were also found to inhibit experimental metastasis in mice and rats [Vlodavsky, I., Mohsen, M., Lider, O., Ishai-Michaeli, R., Ekre, H.-P., Svahn, C. M., Vigoda, M., and Peretz, T. (1995). Inhibition of tumor metastasis by heparanase inhibiting species of heparin. Invasion & Metastasis, 14: 290-302; Nakajima, M., Irimura, T., and Nicolson, G. L. (1988). Heparanase and tumor metastasis. J. Cell. Biochem., 36, 157-167; Parish, C. R., Coombe, D. R., Jakobsen, K. B., and Underwood, P. A. (1987). Evidence that sulfated polysaccharides inhibit tumor metastasis by blocking tumor cell-derived heparanase. Int. J. Cancer, 40, 511-517; Coombe D R, Parish C R, Ramshaw I A, Snowden J M: Analysis of the inhibition of tumor metastasis by sulfated polysaccharides. Int J Cancer 1987; 39:82-8].

Possible Involvement of Heparanase in Tumor Angiogenesis

It was previously demonstrated that heparanase may not only function in cell migration and invasion, but may also elicit an indirect neovascular response [Vlodavsky, I., Bar-Shavit, R., Ishai-Michaeli, R., Bashkin, P., and Fuks, Z. (1991). Extracellular sequestration and release of fibroblast growth factor: a regulatory mechanism? Trends Biochem. Sci., 16, 268-271]. The results suggest that the ECM HSPGs provide a natural storage depot for βFGF and possibly other heparin-binding growth promoting factors. Heparanase mediated release of active βFGF from its storage within ECM may therefore provide a novel mechanism for induction of neovascularization in normal and pathological situations [Vlodavsky, I., Bar-Shavit, R., Korner, G., and Fuks, Z. (1993). Extracellular matrix-bound growth factors, enzymes and plasma proteins. In Basement membranes: Cellular and molecular aspects (eds. D. H. Rohrbach and R. Timpl), pp 327-343. Academic press Inc., Orlando, Fla.; Thunberg L, Backstrom G, Grundberg H, Risenfield J, Lindahl U: The molecular size of the antithrombin-binding sequence in heparin. FEBS Lett 1980; 117:203-206]. However, these prior art references fail to demonstrate the involvement of heparanase in angiogenesis, which therefore still remains to be proved.

Possible Involvement of Heparanase in Wound Healing

Repair of wounds is a chain of processes necessary for removal of damaged tissue or invaded pathogens from the body and for the recovery of the normal skin tissue. The healing process requires a sophisticated interaction between inflammatory cells, biochemical mediators including growth factors, extracellular matrix molecules, and microenvironment cell population. Inflammatory cells, keratinocytes and fibroblasts in the wound space and border produce and release a variety of growth factors such as platelet-derived growth factor (PDGF), epidermal growth factor (EGF), transforming growth factor (TGF) and fibroblast growth factor (FGF). These growth factors have biological activities which stimulate infiltration of inflammatory cells into the wound space and induce proliferation of keratinocytes and fibroblasts, leading to the formation of highly vascularized granulation tissue and extracellular matrix deposition. In deed, topical application of some growth factors (FGF, PDGF) accelerate healing of full-thickness wounds in normal mice and normalize a delayed healing response of diabetic mice [Tsuboi R. and D. B. Rifkin. 1991. Recombinant basic fibroblast growth factor stimulates wound healing-impaired db/db mice. J. Exp. Med. 172: 245-251; Brown R. E., M. P. Breeden and D. G. Greenhalgh. 1994. PDGF and TGF-alpha act synergistically to improve wound healing in the genetically diabetic mouse. J. Surg. Res. 56: 562-570].

Most skin lesions are healed rapidly and efficiently within a week or two. However, the end product is neither aesthetically nor functionally perfect. Moreover, under a number of pathological conditions wound healing is impaired. One such condition is the diabetic state, which result in is a high degree of wound failure, often involved chronic complications including cutaneous infections, immunodisturbance and vascular and neuropathic dysfunction.

Repeated applications of bFGF accelerated closure of full-thickness excisional wounds in diabetic mice. Histological and gross evaluation of wound tissues revealed enhanced angiogenesis in a dose-dependent manner [Okumura M et al; Arzneimittelforschung 1996, 46(10):1021-6]. The angiogenic effect of bFGF was also found to be effective for the treatment of ischemic heart disease and infracted myocardium. In acutely infracted myocardium, bFGF was found to increase the regional myocardial blood flow and salvage the myocardium (rabbit, dog, pig) [Hasegawa T et al; Angiology 1999 50(6):487-95; Scheinowitz M et al; Exp. Physiol. 1998, 83(5):585-93 Miyataka M et al; Angiology 1998, 49(5):381-90]. In addition, bFGF mediated new vessels formation and collateral growth (human, pig, dog) [Watabane E et al; Basic Res. Cardiol. 1998, 93(1):30-7; Fleich M et al; Circulation. 1999, 100(19):1945-50; Yang H T et al; Am. J. Physiol. 1998, 274(6 Pt 2):H2053-61; Schumacher B et al; Circulation. 1998, 97(7):645-50; Arras M et al; J. Clin. Invest. 1998, 101(1):40-50]. bFGF plus heparin was the most effective method of enhancing angiogenesis (pig, dog) ]Uchida Y et al; Am. Heart J. 1995, 130(6):1182-8; Watabane E et al; Basic Res. Cardiol. 1998, 93(1):30-7].

As has already been mentioned above, by degrading HS, heparanase releases a repertoire of effectors such as growth factors from the BM. It may be speculated that the exact repertoire of effectors thus released to a very large extent depends on the specific BM being hydrolyzed.

Relevant Art

U.S. patent application Ser. Nos. 08/922,170; 09/046,475; 09/071,739; 09/071,618; 09/109,386; 09/113,168; 09/140,888; 09/186,200; 09/260,037; 09/258,892; 09/260,038; 09/324,508; 09/322,977; 60/140,801; 09/435,739; 09/487,716; and PCT Application Ser. Nos. US98/17954; US99/06189; US99/09255; US99/09256; US99/15643; US99/25451; US00/03353; US00/03542 are incorporated herein by reference for the sake of providing information regarding the heparanase gene and protein, their alternatives, modifications, other GAG degrading genes and enzymes, their properties, their manufacture and their uses.

Main Objects of the Invention

While reducing the present invention to practice, the ability of heparanase to induce angiogenesis and wound healing were put to test. As is further demonstrates below, the results were striking, rendering heparanase highly likely to become a medication for the induction and/or acceleration of wound healing and/or angiogenesis. Cosmetic applications are envisaged.

### SUMMARY OF THE INVENTION

According to one aspect of the present invention there is provided a method of inducing or accelerating a healing process of a wound, the method comprising the step of administering to the wound a therapeutically effective amount of heparanase, so as to induce or accelerate the healing process of the wound.

According to another aspect of the present invention there is provided a pharmaceutical composition for inducing or accelerating a healing process of a wound, the pharmaceutical composition comprising, as an active ingredient, heparanase and a pharmaceutically acceptable carrier for topical application of the pharmaceutical composition.

According to yet another aspect of the present invention there is provided a method of inducing or accelerating a healing process of a wound, the method compromising the step of implanting into the wound a therapeutically effective amount of heparanase expressing or secreting cells, or heparanase coated cells, so as to induce or accelerate the healing process of the wound.

According to still another aspect of the present invention there is provided a pharmaceutical composition for inducing or accelerating a healing process of a wound, the pharmaceutical composition comprising, as an active ingredient, heparanase expressing or secreting cells, or heparanase coated cells, and a pharmaceutically acceptable carrier being designed for topical application of the pharmaceutical composition.

According to an additional aspect of the present invention there is provided a method of inducing or accelerating a healing process of a wound, the method compromising the step of transforming cells of the wound to produce and secrete heparanase, so as to induce or accelerate the healing process of the wound.

According to yet an additional aspect of the present invention there is provided a pharmaceutical composition for inducing or accelerating a healing process of a wound, the pharmaceutical composition comprising, as an active ingredient, a nucleic acid construct being designed for transforming cells of the wound to produce and secrete heparanase, and a pharmaceutically acceptable carrier being designed for topical application of the pharmaceutical composition.

According to further features in preferred embodiments of the invention described below, the wound is selected from the group consisting of an ulcer, such as a diabetic-ulcer, a burn, a laceration, a surgical incision, necrosis and a pressure wound.

According to still an additional aspect of the present invention there is provided a method of inducing or accelerating angiogenesis, the method comprising the step of administering a therapeutically effective amount of heparanase, so as to induce or accelerate angiogenesis.

According to a further aspect of the present invention there is provided a pharmaceutical composition for inducing or accelerating angiogenesis, the pharmaceutical composition comprising, as an active ingredient, heparanase and a pharmaceutically acceptable carrier.

According to yet a further aspect of the present invention there is provided a method of inducing or accelerating angiogenesis, the method compromising the step of implanting a therapeutically effective amount of heparanase expressing or secreting cells, or heparanase coated cells, so as to induce or accelerate angiogenesis.

According to still a further aspect of the present invention there is provided a pharmaceutical composition for inducing or accelerating angiogenesis, the pharmaceutical composition comprising, as an active ingredient, heparanase expressing or secreting cells, or heparanase coated cells, and a pharmaceutically acceptable carrier.

According to yet another aspect of the present invention there is provided a method of inducing or accelerating angiogenesis, the method compromising the step of transforming cells in vivo to produce and secrete heparanase, so as to induce or accelerate angiogenesis.

According to still another aspect of the present invention there is provided a pharmaceutical composition for inducing or accelerating angiogenesis, the pharmaceutical composition comprising, as an active ingredient, a nucleic acid construct being designed for transforming cells in vivo to produce and secrete heparanase, and a pharmaceutically acceptable carrier.

According to further features in preferred embodiments of the invention described below, the heparanase is contained in a pharmaceutical composition adapted for topical application.

According to still further features in the described preferred embodiments the pharmaceutical composition is packed and identified for treatment of wounds.

According to still further features in the described preferred embodiments the pharmaceutical composition is selected from the group consisting of an aqueous solution, a gel, a cream, a paste, a lotion, a spray, a suspension, a powder, a dispersion, a salve and an ointment.

According to still further features in the described preferred embodiments the pharmaceutical composition includes a solid support.

According to still further features in the described preferred embodiments the heparanase is recombinant.

According to still further features in the described preferred embodiments the heparanase is of a natural source.

According to still further features in the described preferred embodiments the cells are transformed to produce and secrete heparanase.

According to still further features in the described preferred embodiments the cells are transformed by a cis-acting element sequence integrated upstream to an endogenous heparanase gene of the cells and therefore the cells produce and secrete natural heparanase.

According to still further features in the described preferred embodiments the cells are transformed by a recombinant heparanase gene and therefore the cells produce and secrete recombinant heparanase.

According to still further features in the described preferred embodiments the heparanase expressing or secreting cells are capable of forming secretory granules.

According to still further features in the described preferred embodiments the heparanase expressing or secreting cells are endocrine cells.

According to still further features in the described preferred embodiments the heparanase expressing or secreting cells are of a human source.

According to still further features in the described preferred embodiments the heparanase expressing or secreting cells are of a histocompatibility humanized animal source.

According to still further features in the described preferred embodiments the heparanase expressing or secreting cells produce or secrete human heparanase.

According to still further features in the described preferred embodiments the heparanase expressing or secreting cells are autologous cells.

According to still further features in the described preferred embodiments the cells are selected from the group consisting of fibroblasts, epithelial cells, keratinocytes and cells present in a full thickness skin.

The present invention successfully addresses the shortcomings of the presently known configurations by providing new and effective means for inducing or accelerating angiogenesis and wound healing. Cosmetic applications are envisaged.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the Drawings

FIGS. 1a-b demonstrate the expression of heparanase by human endothelium. 1a—RT-PCR. Total RNA isolated from ECGF-stimulated proliferating human umbilical vein (HUVEC, lane 1) and bone marrow (TrHBMEC, lane 2) derived EC was analyzed by RT-PCR for expression of the heparanase mRNA, using human specific hpa primers amplifying a 564 bp cDNA [Vlodavsky, I. et al. Mammalian heparanase: gene cloning, expression and function in tumor progression and metastasis. Nat Med 5, 793-802 (1999)] fragment. Lane 3, DNA molecular weight markers. 1b—Immunohistochemistry. Immunostaining of tissue specimens was performed as described in the Examples section that follows. Positive staining is reddish-brown. Preferential staining of the heparanase protein is seen in the endothelium of capillaries and small sprouting vessels (arrows, left & right panels) as compared to little or no staining of endothelial cells in mature quiescent blood vessels (concave arrows, left & middle panels). A high expression of the heparanase protein is seen in the neoplastic colonic epithelium. Original magnification is 200× (left and right panels) and 100× (middle panel).

FIGS. 2a-c demonstrate release of ECM-bound bFGF by recombinant heparanase, and bFGF accessory activity of HS degradation fragments released from EC vs. ECM. 2a-b—Release of ECM-bound bFGF. 2a ECM-coated wells of four-well plates were incubated (3 hours, 24° C.) with ¹²⁵I-bFGF as described in the Examples section that follows. The ECM was washed 3 times and incubated (3 hours, 37° C.) with increasing concentrations of recombinant heparanase. Released radioactivity is expressed as percent of the total ECM-bound ¹²⁵I-bFGF. About 10% of the ECM-bound ¹²⁵I-bFGF was released in the absence of added heparanase. Each data point is the mean ±SD of triplicate wells. Where error bars cannot be seen, SD is smaller than the symbol. 2a (inset) Release of sulfate labeled HS degradation fragments. Metabolically sulfate labeled ECM was incubated (3 hours, 37° C., pH 6.0) with 0.2 μg/ml recombinant heparanase. Sulfate labeled material released into the incubation medium was analyzed by gel filtration on Sepharose 6B. Labeled fragments eluted in fractions 15-35 (peak II) were 5-6 fold smaller than intact HS side chains and were susceptible to deamination by nitrous acid [Vlodavsky, I. et al. Mammalian heparanase: gene cloning, expression and function in tumor progression and metastasis. Nat Med 5, 793-802 (1999)]. 2b—Release of endogenous ECM-resident bFGF by heparanase. Recombinant heparanase (0.5 μg/ml) was incubated (4 hours, 37° C.) with ECM coated 35-mm dishes in 1 ml heparanase reaction mixture. Aliquots of the incubation media were taken for quantitation of bFGF by ELISA as described in the Examples section that follows. Each data point is the mean±S.D. of triplicate determinations. 2c—Stimulation of bFGF induced DNA synthesis in BaF3 lymphoid cells by HS degradation fragments. Confluent bovine aortic EC cultured in 35-mm plates and their underlying ECM [as described in Gospodarowicz D. Moran J Braun D and Birdwell C 1976 Clonal growth of bovine vascular endothelial cells: fibroblast growth factor as a survival agent. Proc. Natl. Acad. Sci. 73: 4120-4124] were incubated (4 hours, 37° C., pH 6.5) with 0.1 μg/ml recombinant heparanase. Aliquots (5-200 μl) of the incubation media were then added to BaF3 cells seeded into 96 well plates in the presence of 5 ng/ml bFGF. ³H-thymidine (1 μCi/well) was added 48 hours after seeding and 6 hours later the cells were harvested and measured for ³H-thymidine incorporation. Each data point represents the mean ±S.D. of six culture wells. 2c (Inset)—Release of sulfate labeled material from EC (open circles) vs. ECM (closed circles). In control plates, both the EC and ECM were first metabolically labeled with Na₂[³⁵S]O₄. Sulfate labeled material released by heparanase (0.2 μg/ml, 4 hours, 37° C.) from EC and ECM was subjected to gel filtration.

FIGS. 3a-c demonstrate angiogenic response induced by Matrigel embedded with hpa vs. mock transfected Eb lymphoma cells. BALB/c mice (n=5) were injected subcutaneously with 0.4 ml cold Matrigel premixed with 2×10⁶ hpa- or mock-transfected Eb lymphoma cells. After 7 days, the mice were sacrified, and the Matrigel plugs were removed and photographed. Angiogenic response was then quantitated by measurement of the hemoglobin content as described in the Examples section that follows. 3a—Representative Matrigel plugs containing hpa transfected (left) and mock transfected (right) Eb cells photographed in situ, prior to their removal out of their subcutaneous location in the mice. 3b—Matrigel plugs containing heparanase producing (bottom) vs. control mock transfected (top) Eb cells. Shown are isolated Matrigel plugs removed from 10 different mice. 3c—Hemoglobin content of Matrigel plugs (shown in FIG. 3b) containing hpa transfected (dark bar) vs. control mock transfected (empty bar) Eb cells. Represented is the mean ±SD (n=5, p=0.0089; unpaired t test).

FIGS. 4a-b demonstrate that topical administration of active heparanase accelerate wound healing. 4a—Full-thickness wounds were created with a circular 8 mm punch at the back of the mouse skin. Wound areas were calculated after 7 days in control (1) or active heparanase-treated (2) mice and are shown as total area (4a) and percent (4b). Note the enhancement of wound healing upon exogenous application of heparanase. Data are statistically significant (P values equals 0.0023).

FIGS. 5a-d demonstrate an increase in granulation tissue cellularity upon heparanase treatment. Full-thickness wounds were created as described for FIGS. 4a-b. Wounds were left untreated (5a-b) or treated with heparanase for 7 days (5c-d). Wounds, including the underlying granulation tissue were formalin-fixed, paraffin-embedded and 5 micron sections were stained with hematoxilin-eosin. Note the increase in the granulation tissue cellularity upon heparanase treatment. Original magnifications: 4a and 4c×170; 4b and 4d×340.

FIGS. 6a-f demonstrate that heparanase treatment induces cellular proliferation and granulation tissue vascularization. Five micron sections from non-treated (6a, c and d) and heparanase-treated (6b, e and f) granulation tissues were stained for PCNA (6a-b and 6d-e) and for PECAM-1 (6c, f). Note the increase in PCNA-positive cells and PECAM-1 positive blood vessels structures upon heparanase treatment. Original magnifications: 6a-c×170, 6d-f340.

FIGS. 7a-f demonstrates that heparanase expression is restricted to differentiated keratinocytes in mouse skin tissue. Five micron skin tissue sections were stained for PCNA (7a, d) and heparanase (7b-c, e). Negative control (no primary antibody) is shown in 7f. Note intense PCNA staining at the basal epidermal cell layer (7a, d) while heparanase mainly stain the outer most, keratinocytes, cell layer (7b, e) and the cells composing the hair follicle (7c). In the latter case, nuclear staining is observed.

FIGS. 8a-d demonstrate expression of heparanase in human skin. 8a—cultures of HaCat keratinocytes cell line immunostained with antiheparanase monoclonal antibody (HP-92). 8b—heparanase activity in intact cells and in extracts of HaCat cells, in an ECM-assay. 8c and d—immunostaining of normal skin tissue with HP-92.

FIG. 9 demonstrates stimulation of angiogenesis by heparanase in rat eye model. The central cornea of rats' eyes was scraped with a surgical knife. The right eye of each rat was then treated with heparanase, 50 μl drop (1 mg/ml) of purified recombinant human P50 heparanase, three times a day. The left eye served as a control and was treated with Lyeteers. Vascularization and epithelialization were evaluated following closure of the corneal lesion. Heparanase treated eyes exhibited vascularization of the cornea, as well as increased vascularization in the iris. Normal, minor vascularization of the iris and non vascular appearance of the cornea were observed in the controls

FIG. 10 demonstrates cornea sections of heparanase treated eye as compared to control, Lyeteers treated eyes. Control eyes demonstrate healing of the epithelia which is accompanied by a normal organized structure of the cornea. Heparanase treatment resulted in growth of blood vessels into the cornea (arrows), followed by a massive infiltration of lymphocytes. Vascularization associated inflammatory reaction interfered with corneal healing, as demonstrated by a disorganized structure of the cornea.

### DESCRIPTION OF THE PREFERRED EMBODIMENTS

The present invention is of methods and compositions which can be used for inducing and/or accelerating wound healing and/or angiogenesis, as well as for cosmetic treatment of hair and skin.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Extracellular matrix (ECM) and in particular basement membranes (BM) present a main physical barrier which requires enzymatic degradation during endothelial cell sprouting at early stages of angiogenesis [Hanahan, D. & Folkman, J. Patterns and emerging mechanisms of the angiogenic switch during tumorogenesis. Cell 86, 353-364 (1996)]. These multi-molecular structures also serve as a storage depot for heparin-binding angiogenic growth factors [Vlodavsky, I., Bar-Shavit, R., Korner, G. & Fuks, Z. Extracellular matrix-bound growth factors, enzymes and plasma proteins. In Basement membranes: Cellular and molecular aspects (eds. D. H. Rohrbach and R. Timpl), Academic Press Inc., Orlando, Fla., pp 327-343, (1993)]. Heparan sulfate proteoglycans (HSPGs) are responsible for the self-assembly and integrity of the ECM and BM structure, as well as for binding and sequestration of growth and differentiation factors [Bemfield, M. et al. Functions of cell surface heparan sulfate proteoglycans. Annu Rev Biochem 68, 729-777 (1999); lozzo, R. V. & Murdoch, A. D. Proteoglycans of the extracellular environment: clues from the gene and protein side offer novel perspectives in molecular diversity and function. FASEB J. 10, 598-614 (1996)]. Recently, the cloning of heparanase, an endo-β-Dglucuronidase degrading heparan sulfate (HS), was reported and a direct evidence for its role in tumor invasion and metastasis was provided [Vlodavsky, I. et al. Mammalian heparanase: gene cloning, expression and function in tumor progression and metastasis. Nat Med 5, 793-802 (1999)]. It is demonstrated herein for the first time that heparanase is tightly involved in angiogenesis and its mode of action elucidated. Apart from its direct involvement in ECM degradation and endothelial cell migration (vascular sprouting), hepatanase releases active bFGF from the subendothelial ECM, as well as bFGF-stimulating HS degradation fragments from the endothelial cell surface. Interestingly, HS fragments released from ECM do not potentiate the growth promoting activity of bFGF. The conclusive angiogenic potential of heparanase was demonstrated in vivo (Matrigel plug assay) by showing a 3-4-fold increase in neovascularization induced by Eb murine T-lymphoma cells following stable transfection with the heparanase gene. Immunohistochemical staining of human colon carcinoma tissue revealed a high expression of the heparanase protein in the endothelium of sprouting capillaries, but not of mature quiescent vessels in the same tissue section. The ability of heparanase to promote tumor angiogenesis together with its involvement in tumor invasiveness and metastasis make it a promising target for cancer therapy.

HSPGs are most abundant in cell surfaces, ECM and BM [Bemfield, M. et al. Functions of cell surface heparan sulfate proteoglycans. Annu Rev Biochem 68, 729-777 (1999); lozzo, R. V. & Murdoch, A. D. Proteoglycans of the extracellular environment: clues from the gene and protein side offer novel perspectives in molecular diversity and function. FASEB J. 10, 598-614 (1996)]. BM represents specialized ECM structures which underlay endothelial cells (EC) in the blood vessel wall, as well as epithelial cells in various tissues and organs. HSPGs, the major polysaccharide-containing component of BM, play a key role in the self-assembly and integrity of the BM multimolecular architecture. This function is clearly ascribed to the HS carbohydrate side chains [Bemfield, M. et al. Functions of cell surface heparan sulfate proteoglycans. Annu Rev Biochem 68, 729-777 (1999); lozzo, R. V. & Murdoch, A. D. Proteoglycans of the extracellular environment: clues from the gene and protein side offer novel perspectives in molecular diversity and function. FASEB J. 10, 598-614 (1996)]. HS chains interact through specific attachment sites with the main protein components of the ECM and BM, such as collagen IV, laminin and fibronectin, thus contributing to the integrity of the BM structure. Recently, it is becoming increasingly clear that HSPGs are also actively involved in orchestrating cellular responses in both normal and pathological conditions [Bemfield, M. et al. Functions of cell surface heparan sulfate proteoglycans. Annu Rev Biochem 68, 729-777 (1999); lozzo, R. V. & Murdoch, A. D. Proteoglycans of the extracellular environment: clues from the gene and protein side offer novel perspectives in molecular diversity and function. FASEB J. 10, 598-614 (1996)], ranging from pregnancy and development to neovascularization and metastatic spread of malignant tumors.

The importance of HS and in particular its enzymatic degradation during angiogenesis attracted a growing attention during the last decade. Angiogenesis represents a coordinated multicellular process that requires the functional activity of a wide variety of molecules, including growth factors, ECM components, adhesion receptors, and matrix-degrading enzymes [Hanahan, D. & Folkman, J. Patterns and emerging mechanisms of the angiogenic switch during tumorogenesis. Cell 86, 353-364 (1996)]. HS and HS-degrading enzymes are implicated in a number of angiogenesisrelated cellular events, such as cell invasion, migration, adhesion, differentiation and proliferation [Bemfield, M. et al. Functions of cell surface heparan sulfate proteoglycans. Annu Rev Biochem 68, 729-777 (1999); lozzo, R. V. & Murdoch, A. D. Proteoglycans of the extracellular environment: clues from the gene and protein side offer novel perspectives in molecular diversity and function. FASEB J. 10, 598-614 (1996)].

An important early event in the angiogenic cascade is degradation of the subendothelial BM by proliferating EC and formation of vascular sprouts [Hanahan, D. & Folkman, J. Patterns and emerging mechanisms of the angiogenic switch during tumorogenesis. Cell 86, 353-364 (1996); Stetler-Stevenson, W. G. Matrix metalloproteinases in angiogenesis: a moving target for therapeutic intervention. J Clin Invest 103, 1237-1241 (1999)]. Enzymatic cleavage of HS, the polysaccharide scaffold of BM, is believed to contribute significantly to the invasive ability of EC and their subsequent migration through the ECM toward the angiogenic stimulus.

Several species of HSPGs are not secreted into the ECM, but rather are found on the cell surface [Bernfield, M. et al. Functions of cell surface heparan sulfate proteoglycans. Annu Rev Biochem 68, 729-777 (1999)]. Transmembrane and membrane anchored HSPGs have a co-receptor role in which the HS, in concert with tyrosine kinase signaling receptors comprise a functional complex that binds various members of the heparin-binding growth factor family, of which basic fibroblast growth factor (bFGF) and vascular endothelial growth factor (VEGF) are regarded as the two major proangiogenic molecules [Hanahan, D. & Folkman, J. Patterns and emerging mechanisms of the angiogenic switch during tumorogenesis. Cell 86, 353-364 (1996); Bemfield, M. et al. Functions of cell surface heparan sulfate proteoglycans. Annu Rev Biochem 68, 729-777 (1999); SpivakKroizman, T. et al. Heparin-induced oligomerization of FGF molecules is responsible for FGF receptor dimerization, activation, and cell proliferation. Cell 79, 1015-1024 (1994); Vlodavsky, I., Miao, H. Q., Medalion, B., Danagher, P. & Ron, D. 1996. Involvement of heparan sulfate and related molecules in sequestration and growth promoting activity of fibroblast growth factor. Cancer Metastasis Rev 15, 177-186 (1996); Aviezer, D. et al. Perlecan, basal lamina proteoglycan, promotes basic fibroblast growth factor-receptor binding, mitogenesis, and angiogenesis. Cell 79, 1005-1013 (1994)].

Interactions of HS with bFGF were studied extensively. Basic FGF requires HS as a cofactor for signaling. Cell surface HS bearing specific saccharide sequences function as accessory co-receptors for bFGF, facilitating high affinity receptor binding, inducing bFGF-receptor dimerization, autophosphorylation and signaling [Spivak-Kroizman, T. et al. Heparin-induced oligomerization of FGF molecules is responsible for FGF receptor dimerization, activation, and cell proliferation. Cell 79, 1015-1024 (1994); Vlodavsky, I., Miao, H. Q., Medalion, B., Danagher, P. & Ron, D. 1996. Involvement of heparan sulfate and related molecules in sequestration and growth promoting activity of fibroblast growth factor. Cancer Metastasis Rev 15, 177-186 (1996); Aviezer, D. et al. Perlecan, basal lamina proteoglycan, promotes basic fibroblast growth factor-receptor binding, mitogenesis, and angiogenesis. Cell 79, 1005-1013 (1994); Miao, H. Q., Omitz, D. M., Aingorn, E., Ben-Sasson, S. A. & Vlodavsky, I. Modulation of fibroblast growth factor-2 receptor binding, dimerization, signaling, and angiogenic activity by a synthetic heparin-mimicking polyanionic compound. J Clin Invest 99, 1565-1575 (1997)].

ECM- and BM-resident HSPGs appear to be less active than cell surface HS in mediating bFGF/FGF-receptor complex assembly and function [Chang, Z., Meyer, K., Rapraeger, A. C. & Friedl, A. Differential ability of heparan sulfate proteoglycans to assemble the fibroblast growth factor receptor complex in situ. FASEB J 14, 137-144 (2000)]. Rather, they bind specifically bFGF and serves as its extracellular reservoir [Vlodavsky, I., Bar-Shavit, R., Korner, G. & Fuks, Z. Extracellular matrix-bound growth factors, enzymes and plasma proteins. In Basement membranes: Cellular and molecular aspects (eds. D. H. Rohrbach and R. Timpl), Academic Press Inc., Orlando, Fla., pp 327-343, (1993); Vlodavsky, I., Miao, H. Q., Medalion, B., Danagher, P. & Ron, D. 1996. Involvement of heparan sulfate and related molecules in sequestration and growth promoting activity of fibroblast growth factor. Cancer Metastasis Rev 15, 177-186 (1996)]. ECM sequestration of bFGF by HSPGs is well documented. Basic FGF was extracted from the subendothelial ECM in vitro and from both endothelial and epithelial BM of the cornea [Vlodavsky, I., Bar-Shavit, R., Korner, G. & Fuks, Z. Extracellular matrix-bound growth factors, enzymes and plasma proteins. In Basement membranes: Cellular and molecular aspects (eds. D. H. Rohrbach and R. Timpl), Academic Press Inc., Orlando, Fla., pp 327-343, (1993); Vlodavsky, I., Miao, H. Q., Medalion, B., Danagher, P. & Ron, D. 1996. Involvement of heparan sulfate and related molecules in sequestration and growth promoting activity of fibroblast growth factor. Cancer Metastasis Rev 15, 177-186 (1996)]. Similarly, bFGF is distributed ubiquitously in BM of all size blood vessels [Vlodavsky, I., Bar-Shavit, R., Korner, G. & Fuks, Z. Extracellular matrix-bound growth factors, enzymes and plasma proteins. In Basement membranes: Cellular and molecular aspects (eds. D. H. Rohrbach and R. Timpl), Academic Press Inc., Orlando, Fla., pp 327-343, (1993)]. Despite the ubiquitous presence of bFGF in normal tissues, EC proliferation in these tissues is usually very low; suggesting that bFGF is sequestered from its site of action [Vlodavsky, I., Bar-Shavit, R., Korner, G. & Fuks, Z. Extracellular matrix-bound growth factors, enzymes and plasma proteins. In Basement membranes: Cellular and molecular aspects (eds. D. H. Rohrbach and R. Timpl), Academic Press Inc., Orlando, Fla., pp 327-343, (1993); Vlodavsky, I., Miao, H. Q., Medalion, B., Danagher, P. & Ron, D. 1996. Involvement of heparan sulfate and related molecules in sequestration and growth promoting activity of fibroblast growth factor. Cancer Metastasis Rev 15, 177-186 (1996)].

It appears that HS moieties are specific for binding and sequestration of bFGF in BM, as other glycosaminoglycans (i.e., chondroitin sulfate, dermatan sulfate, keratan sulfate) do not bind bFGF. In support of specific binding of bFGF to HS is the observation that up to 90% of the bound growth factor was displaced by heparin or HS [Vlodavsky, I., Bar-Shavit, R., Korner, G. & Fuks, Z. Extracellular matrix-bound growth factors, enzymes and plasma proteins. In Basement membranes: Cellular and molecular aspects (eds. D. H. Rohrbach and R. Timpl), Academic Press Inc., Orlando, Fla., pp 327-343, (1993); Vlodavsky, I., Miao, H. Q., Medalion, B., Danagher, P. & Ron, D. 1996. Involvement of heparan sulfate and related molecules in sequestration and growth promoting activity of fibroblast growth factor. Cancer Metastasis Rev 15, 177-186 (1996)]. It is conceivable that an enzyme such as heparanase degrading HS could be a most effective specific releaser of ECM-resident bFGF. Therefore, apart of direct involvement in BM invasion by endothelial cells (EC), degradation of HS may elicit an indirect angiogenic response by releasing HS-bound angiogenic growth factors (e.g., bFGF, VEGF) from ECM and BM [Vlodavsky, I., Bar-Shavit, R., Korner, G. & Fuks, Z. Extracellular matrix-bound growth factors, enzymes and plasma proteins. In Basement membranes: Cellular and molecular aspects (eds. D. H. Rohrbach and R. Timpl), Academic Press Inc., Orlando, Fla., pp 327-343, (1993); Vlodavsky, I., Miao, H. Q., Medalion, B., Danagher, P. & Ron, D. 1996. Involvement of heparan sulfate and related molecules in sequestration and growth promoting activity of fibroblast growth factor. Cancer Metastasis Rev 15, 177-186 (1996)] and by generating HS fragments which can potentiate bFGF receptor binding, dimerization and signaling [Spivak-Kroizman, T. et al. Heparin-induced oligomerization of FGF molecules is responsible for FGF receptor dimerization, activation, and cell proliferation. Cell 79, 1015-1024 (1994); Vlodavsky, I., Miao, H. Q., Medalion, B., Danagher, P. & Ron, D. 1996. Involvement of heparan sulfate and related molecules in sequestration and growth promoting activity of fibroblast growth factor. Cancer Metastasis Rev 15, 177-186 (1996); Aviezer, D. et al. Perlecan, basal lamina proteoglycan, promotes basic fibroblast growth factor-receptor binding, mitogenesis, and angiogenesis. Cell 79, 1005-1013 (1994)].

Based on these considerations, the potential involvement of heparanase in neovascularization, both in vitro and in vivo was investigated. Endoglycosidic heparanase, degrading HS side chains of HSPGs, has been studied for its role in tumor progression during the last two decades [Vlodavsky, I. et al. Inhibition of tumor metastasis by heparanase inhibiting species of heparin. Invasion Metastasis 14, 290-302 (1994)], but only recently the mammalian heparanase gene was cloned [Vlodavsky, I. et al. Mammalian heparanase: gene cloning, expression and function in tumor progression and metastasis. Nat Med 5, 793-802 (1999); Hulett, M. D. et al. Cloning of mammalian heparanase, an important enzyme in tumor invasion and metastasis. Nat Med 5, 803-809 (1999)] and provided the first direct evidence for its role in tumor invasion and metastasis [Vlodavsky, I. et al. Mammalian heparanase: gene cloning, expression and function in tumor progression and metastasis. Nat Med 5, 793-802 (1999)]. In the present study, the availability of recombinant enzyme, specific antibodies and molecular probes enabled us to demonstrate a causative involvement of the heparanase enzyme in tumor-associated angiogenesis and to elucidate its mode of action.

While reducing one aspect of the present invention to practice, the expression of heparanase by vascular EC in vitro and in angiogenic blood vessels was studied. Previously, it has been suggested that stimulated EC secrete heparanase-like activity [Godder, K. et al. Heparanase activity in cultured endothelial cells. J Cell Physiol 148, 274-280 (1991); Pillarisetti, S. et al. Endothelial cell heparanase modulation of lipoprotein lipase activity. Evidence that heparan sulfate oligosaccharide is an extracellular chaperone. J Biol Chem 272, 15753-15759 (1997)]. Using RT-PCR, it is now unequivocally demonstrates, for the first time, that the heparanase gene is expressed by proliferating human EC. Both cultured human umbilical vein EC (HUVEC) and human bone marrow EC (TrHBMEC) [Schweitzer, K. M. et al. Characterization of a newly established human bone marrow endothelial cell line: distinct adhesive properties for hematopoietic progenitors compared with human umbilical vein endothelial cells. Lab Invest 76, 25-36 (1997)] expressed the heparanase gene. Staining paraffin embedded sections from patients with primary colon adenocarcinoma with monoclonal anti-heparanase antibodies revealed that the heparanase protein is preferentially expressed in sprouting capillaries whereas the endothelium of mature quiescent vessels showed no detectable levels of heparanase. A similar expression pattern was observed in human mammary and pancreatic carcinomas, suggesting a significant role of endothelial heparanase in enabling EC to traverse BM and ECM barriers during sprouting angiogenesis. As previously reported [Vlodavsky, I. et al. Mammalian heparanase: gene cloning, expression and function in tumor progression and metastasis. Nat Med 5, 793-802 (1999)] and also demonstrated herein, the neoplastic colonic mucosa exhibits an intense heparanase staining, as opposed to no expression of heparanase in normal colon epithelium [Vlodavsky, I. et al. Mammalian heparanase: gene cloning, expression and function in tumor progression and metastasis. Nat Med 5, 793-802 (1999)]. Carcinoma cells can therefore be regarded as the main source of heparanase in the tumor microenvironment. Moreover, at a later stage of tumor progression, heparanase was also found in the tumor stroma.

A straightforward explanation for the role of tumor- and stroma-derived heparanase in angiogenesis is release of ECM-resident bFGF and other heparin-binding angiogenic factors [Vlodavsky, I., Bar-Shavit, R., Komer, G. & Fuks, Z. Extracellular matrix-bound growth factors, enzymes and plasma proteins. In Basement membranes: Cellular and molecular aspects (eds. D. H. Rohrbach and R. Timpl), Academic Press Inc., Orlando, Fla., pp 327-343, (1993); Vlodavsky, I., Miao, H. Q., Medalion, B., Danagher, P. & Ron, D. 1996. Involvement of heparan sulfate and related molecules in sequestration and growth promoting activity of fibroblast growth factor. Cancer Metastasis Rev 15, 177-186 (1996)]. As is shown in the Examples section below, degradation of HS in the ECM resulted in release of as much as 70% of the ECM-bound bFGF. In another experiment it is shown that released bFGF stimulates 5-8 fold the proliferation of 3T3 fibroblasts and bovine aortic EC. These results clearly indicate that heparanase releases active bFGF sequestered as a complex with HS in the ECM. Both tumor and endothelial heparanase may hence elicit an indirect angiogenic response by means of releasing active HS-FGF complexes from storage in the ECM and tumor microenvironment.

The ability of heparanase cleaved HS degradation fragments to promote the mitogenic activity of bFGF was investigated using a cytokine-dependent lymphoid cell line (BaF3, clone 32) engineered to express FGF-receptor 1 (FGFR1) [Miao, H. Q., Ornitz, D. M., Aingorn, E., Ben-Sasson, S. A. & Vlodavsky, I. Modulation of fibroblast growth factor-2 receptor binding, dimerization, signaling, and angiogenic activity by a synthetic heparin-mimicking polyanionic compound. J Clin Invest 99, 1565-1575 (1997); Ornitz, D. M. et al. Heparin is required for cell-free binding of basic fibroblast growth factor to a soluble receptor and for mitogenesis in whole cells. Mol Cell Biol 12, 240-247 (1992)]. The results indicate that the heparanase enzyme potentiates the mitogenic activity of bFGF and possibly other heparin-binding angiogenic growth factors, through release of HS degradation fragments that promote bFGF-receptor binding and activation. The observed difference in biological activity between cell surface- and ECM-derived HS fragments indicates that the primary role of HS in the ECM is to sequester, protect and stabilize heparin-binding growth factors, while the cell surface HS plays a more active role in promoting the mitogenic and angiogenic activities of the growth factor by means of stimulating receptor binding, dimerization and activation. This concept is supported by the recently reported preferential ability of cell surface- vs. ECM-HSPG to mediate the assembly of bFGF-receptor signaling complex [Chang, Z., Meyer, K., Rapraeger, A. C. & Friedl, A. Differential ability of heparan sulfate proteoglycans to assemble the fibroblast growth factor receptor complex in situ. FASEBJ 14, 137-144 (2000)].

The Matrigel plug assay [Passaniti, A. et al. A simple, quantitative method for assessing angiogenesis and antiangiogenic agents using reconstituted basement membrane, heparin, and fibroblast growth factor. Lab Invest 67, 519-528 (1992)] was applied to investigate whether the heparanase enzyme can elicit an angiogenic response in vivo. A pronounced angiogenic response was induced by Matrigel embedded Eb cells over expressing the heparanase enzyme, as compared to little or no neovascularization exerted by mock transfected Eb cells expressing no heparanase activity. The angiogenic response was reflected by a network of capillary blood vessels attracted toward the Matrigel plug containing heparanase transfected vs. control mock transfected Eb cells, and by a large amount of blood and vessels seen in the isolated Matrigel plugs excised from each of the mice. This difference was highly significant, as also demonstrated by measurements of the hemoglobin content of Matrigel plugs removed from each mouse of the respective groups.

These findings, together with previous results on the increased metastatic potential of heparanase transfected vs. mock transfected Eb cells [Vlodavsky, I. et al. Mammalian heparanase: gene cloning, expression and function in tumor progression and metastasis. Nat Med 5, 793-802 (1999)] emphasize the significance of heparanase in the two critical events in tumor progression: metastasis and angiogenesis.

Compounds that inhibit the heparanase enzyme are therefore anticipated to exert an anti-cancerous effect through inhibition of both tumor cell metastasis [Vlodavsky, I. et al. Mammalian heparanase: gene cloning, expression and function in tumor progression and metastasis. Nat Med 5, 793-802 (1999); Vlodavsky, I. et al. Inhibition of tumor metastasis by heparanase inhibiting species of heparin. Invasion Metastasis 14, 290-302 (1994)] and angiogenesis.

The primary, goal in the treatment of wounds is to achieve wound closure. Open cutaneous wounds represent one major category of wounds and include burn wounds, neuropathic ulcers, pressure sores, venous stasis ulcers and diabetic ulcers. Open cutaneous wounds routinely heal by a process which comprises six major components: (i) inflammation; (ii) fibroblast proliferation; (iii) blood vessel proliferation; (iv) connective tissue synthesis; (v) epithelialization; and (vi) wound contraction. Wound healing is impaired when these components, either individually or as a whole, do not function properly. Numerous factors can affect wound healing, including malnutrition, infection, pharmacological agents (e.g., actinomycin and steroids), advanced age immunodeficiency and diabetes [see Hunt and Goodson in Current Surgical Diagnosis & Treatment (Way; Appleton & Lange), pp. 86-98 (1988)].

With respect to diabetes, diabetes mellitus is characterized by impaired insulin signaling, elevated plasma glucose and a predisposition to develop chronic complications involving several distinctive tissues. Among all the chronic complications of diabetes mellitus, impaired wound healing leading to foot ulceration is among the least well studied. Yet skin ulceration in diabetic patients takes a staggering personal and financial cost [Knighton, D. R. and Fiegel, V. D. Growth factors and comprehensive surgical care of diabetic wounds. Curr. Opin. Gen. Surg.:32-9: 32-39, 1993; Shaw, J. E. and Boulton, A. J. The pathogenesis of diabetic foot problems: an overview. Diabetes, 46 Suppl 2: S58-S61, 1997].

Moreover, foot ulcers and the subsequent amputation of a lower extremity are the most common causes of hospitalization among diabetic patients [Shaw, J. E. and Boulton, A. J. The pathogenesis of diabetic foot problems: an overview. Diabetes, 46 Suppl 2:S58-61: S58-S611997; Coghlan, M. P., Pillay, T. S., Tavare, J. M., and Siddle, K. Site-specific anti-phosphopeptide antibodies: use in assessing insulin receptor serine/threonine phosphorylation state and identification of serine-1327 as a novel site of phorbol ester-induced phosphorylation. Biochem.J., 303: 893-899, 1994; Grunfeld, C. Diabetic foot ulcers: etiology, treatment, and prevention. Adv. Intern. Med. 37.103-32: 103-132, 1992; Reiber, G. E., Lipsky, B. A., and Gibbons, G. W. The burden of diabetic foot ulcers. Am. J. Surg., 176: 5S-10S, 1998]. In diabetes, the wound healing process is impaired and healed wounds are characterized by diminished wound strength.

Skin is a stratified squamous epithelium in which cells undergoing growth and differentiation are strictly compartmentalized. In the physiologic state, proliferation is confined to the basal cells that adhere to the basement membrane. Differentiation is a spatial process where basal cells lose their adhesion to the basement membrane, cease DNA synthesis and undergo a series of morphological and biochemical changes. The ultimate maturation step is the production of the cornified layer forming the protective barrier of the skin [Hennings, H., Michael, D., Cheng, C., Steinert, P., Holbrook, K., and Yuspa, S. H. Calcium regulation of growth and differentiation of mouse epidermal cells in culture. Cell, 19: 245-254, 1980; Yuspa, S. H., Kilkenny, A. E., Steinert, P. M., and Roop, D. R. Expression of murine epidermal differentiation markers is tightly regulated by restricted extracellular calcium concentrations in vitro. J. Cell Biol., 109: 1207-1217, 1989].

The earliest changes observed when basal cells commit to differentiate is associated with the ability of the basal cells to detach and migrate away from the basement membrane [Fuchs, E. Epidermal differentiation: the bare essentials. J. Cell Biol., 111: 2807-2814, 1990.]. Similar changes are associated with the wound healing process where cells both migrate into the wound area and proliferative capacity is enhanced. These processes are mandatory for the restructuring of the skin layers and induction of proper differentiation of the epidermal layers.

Adult skin includes two layers: a keratinized stratified epidermis and an underlying thick layer of collagen-rich dermal connective tissue providing support and nourishment. Skin serves as the protective barrier against the outside world. Therefore any injury or break in the skin must be rapidly and efficiently mended. As described hereinabove, the first stage of the repair is achieved by formation of the clot that plugs the initial wound. Thereafter, inflammatory cells, fibroblasts and capillaries invade the clot to form the granulation tissue. The following stages involve re-epithelization of the wound where basal keratinocytes have to lose their hemidesmosomal contacts, keratinocytes migrate upon the granulation tissue to cover the wound. Following keratinocyte migration, keratinocytes enter a proliferative boost, which allows replacement of cells lost during the injury. After the wound is covered by a monolayer of keratinocytes, new stratified epidermis is formed and the new basement membrane is reestablished [Weinstein, M. L. Update on wound healing: a review of the literature. Mil. Med., 163: 620-624, 1998; Singer, A. J. and Clark, R. A. Cutaneous wound healing. N. Engl. J. Med., 341: 738-746, 1999; Whitby, D. J. and Ferguson, M. W. Immunohistochemical localization of growth factors in fetal wound healing. Dev. Biol., 147: 207-215, 1991; Kiritsy, C. P., Lynch, B., and Lynch, S. E. Role of growth factors in cutaneous wound healing: a review. Crit. Rev. Oral Biol. Med., 4: 729-760, 1993].

Several growth factors have been shown to participate in this process is including EGF family of growth factors, KGF, PDGF and TGFβ1 [Whitby, D. J. and Ferguson, M. W. Immunohistochemical localization of growth factors in fetal wound healing. Dev. Biol., 147: 207-215, 1991; Kiritsy, C. P., Lynch, .B., and Lynch, S. E. Role of growth factors in cutaneous wound healing: a review. Crit. Rev. Oral Biol. Med., 4: 729-760, 1993; Andresen, J. L., Ledet, T., and Ehlers, N. Keratocyte migration and peptide growth factors: the effect of PDGF, bFGF, EGF, IGF-I, aFGF and TGF-beta on human keratocyte migration in a collagen gel. Curr. Eye Res., 16: 605-613, 1997]. Among these growth factors both EGF and KGF are thought to be intimately involved in the regulation of proliferation and migration of epidermal keratinocytes [Werner, S., Breeden, M., Hubner, G., Greenhalgh, D. G., and Longaker, M. T. Induction of keratinocyte growth factor expression is reduced and delayed during wound healing in the genetically diabetic mouse. J. Invest. Dermatol., 103: 469-473, 1994; Threadgill, D. W., Dlugosz, A. A., Hansen, L. A., Tennenbaum, T., Lichti, U., Yee, D., LaMantia, C., Mourton, T., Herrup, K., Harris, R. C., Barnard, J. A., Yuspa, S. H., Coffey, R. J., and Magnuson, T. Targeted disruption of mouse EGF receptor: effect of genetic background on mutant phenotype. Science, 269: 230-234, 1995].

As has already been mentioned hereinabove, heparan sulfate proteoglycan (HSPGs) are ubiquitous macromolecules associated with the cell surface and the extracellular matrix (ECM). The ability of heparan sulfate to interact with ECM molecules such as collagen, laminin and fibronectin indicates that this proteoglycan is essential for self-assembly, insolubility and function of the ECM. Initially envisioned as a physical tissue support, it is now clear that the ECM actively transmit biochemical signals, which affect a variety of cellular behaviors. These include cell adhesion, proliferation, migration, survival, locomotion and tissue integrity, function, morphology and architecture. Expression of HS-degrading endoglycosidases, commonly called heparanases, correlates with the metastatic potential of mouse and human lymphoma, fibrosarcoma, and melanoma cell lines, and with extravasation associated with inflammation and autoimmunity. In addition to being involved in the remodeling of ECM and egress of cells from the vasculature, heparanase may regulate angiogenesis, tissue repair and remodeling as well as wound healing by releasing HS-bound growth factors (e.g., bFGF, KGF, VEGF, HGF, HB-EGF), cytokines [interleukin (IL) 1, 8, 10] and chemokines (RANTES, MCP-1, MIP 1; [Vaday G. G. and O. Lider. 2000. Extracellular matrix moieties, cytokine, and enzymes: dynamic effect on immune cell behavior and inflammation. J. Leukoc. Biol. 67: 149-159]). The release of such proteins associated with low molecular weight HS can potentiate the interaction of soluble growth factors with their cell surface receptors, as has been shown for bFGF [Vlodavsky I., H.-Q. Miao, B. Medalion, P. Danagher and D. Ron. 1996. Involvement of heparan sulfate and related molecules in sequestration and growth promoting activity of fibroblast growth factor. Cancer and Metastasis Reviews 15: 177-186], or can protect the bound protein from proteolytic cleavage.

Until recently, the nature of heparanase was a matter of dispute. However, within the past two years, several laboratories have purified human heparanase and isolated the cDNA encoding this activity [Vlodavsky I., Y. Friedman, M. Elkin, H. Aingorn, R. Atzmon, R. Ishai-Michaeli, M. Bitan, O. Pappo, T. Peretz, I. Michal, L. Spector and I. Pecker. 1999. Mammalian heparanase: Gene cloning, expression and function in tumor progression and metastasis. Nature Med. 5: 793-802; Hulett M. D., C. Freeman, B. J. Hamdorf, R. T. Baker, M. J. Harris and C. R. Parish. 1999. Cloning of mammalian heparanase, an important enzyme in tumor invasion and metastasis. Nature Med. 5: 803-809; Toyoshima M. and M. Nakajima. 1999. Human heparanase: purification, characterization, cloning and expression. J. Biol. Chem. 274: 24153-24160]. Expression of the cloned cDNA in insect and mammalian cells yielded 65 and 50 kDa glycoproteins. The 50 kDa enzyme represent an N-terminal processed enzyme, which is at least 200-fold more active than the full-length 65 kDa protein [Vlodavsky I., Y. Friedman, M. Elkin, H. Aingom, R. Atzmon, R. Ishai-Michaeli, M. Bitan, O. Pappo, T. Peretz, I. Michal, L. Spector and I. Pecker. 1999. Mammalian heparanase: Gene cloning, expression and function in tumor progression and metastasis. Nature Med. 5: 793-802]. Heparanase activities purified from different human and animal sources are related immunologically, share substrate specificities, yield similar oligosaccharide cleavage products and are inhibited by heparin substrate derivatives. This may suggest that the cloned enzyme represent the predominant heparanase in mammalian species. The availability of purified active enzyme made it possible to further explore the role of heparanase in a highly controlled manner and in a specific biological setting.

While reducing one aspect of the present invention to practice it was demonstrated that the active 50 kDa heparanase enzyme accelerate wound closure in a mouse skin model.

Indirect evidences correlated heparanase activity to angiogenesis and inflammation, which are both required for successful wound healing.

In order to directly study the effect of heparanase on the complex of events composing wound healing, active heparanase was applied topically onto full-thickness wounds. Careful evaluation of wounds areas revealed a significant improvement of wound closure upon heparanase treatment.

It is known that the inactive form of heparanase, P60, is activatable in vivo, via proteolysis into its active form P50 (see, for example, U.S. patent application Ser. No. 09/260,037), and may therefore also be used in accordance with the teachings of the present invention for wound healing, induction of angiogenesis and/or for cosmetic applications.

Having demonstrated, for the first time, a direct role for heparanase activity in the wound healing process, cellular and molecular mechanisms that are activated by heparanase in the course of wound healing were sought for. Examination of hematoxilin-eosin stained wound sections revealed the expected granulation tissue morphology, composed of fibroblasts, blood vessels and inflammatory cells. Interestingly, the heparanase-treated granulation tissue was much more dense. Specifically, a significant increase in the number of inflammatory cells and blood vessels was observed. This was further confirmed by staining for PCNA, a marker for cell proliferation and for PECAM-1, a marker for endothelial cells. Indeed, an increase in PCNA and PECAM-1 staining was observed in the granulation tissue of heparanase-treated wounds. Thus, the acceleration of wound healing is, without limitation, due to the robust fibroblast and inflammatory cells-derived cytokine and chemokines and to increased vascularity. Heparanase was found to be expressed by all the major cell components of granulation tissue. Interestingly, heparanase expression was mainly detected in the differentiated, non-proliferating, cells composing the epidermis, while proliferating, PCNA-positive epidermal cells reconstituting the wound were poorly stained. In addition, heparanase staining was observed in nonproliferating hair follicle cells. Such staining pattern suggests, without limitation, that heparanase plays a role in cellular terminal differentiation which leads, as in the case of keratinocyes, to apoptosis and as an antiinfectant.

Heparan sulfates are prominent components of blood vessels. In capillaries they are found mainly in the subendothelial basement membrane, supporting and stabilizing the structure of blood vessels wall. Cleavage of the underlying ECM plays a decisive part not only in the extravasation of blood-born (immune) cells, but also in the sprouting of new capillaries from pre-existing blood vessels. This early step is believed to contribute significantly to the invasive ability of endothelial cells and their subsequent migration through the ECM toward the angiogenic stimulus. Heparanase expression was detected in -proliferating endothelial cells in vitro and, moreover, in sprouting capillaries in vivo. In contrast, the endothelium of mature, quiescent vessels showed no detectable heparanase expression, suggesting that heparanase activity may be involved in angiogenic sprout formation.

Wounded skin will cause leakage of blood from damaged blood vessels and the formation of fibrin clot. Importantly, the clot serves as a reservoir for cytokines and growth factors that are released as activated platelets degranulate [Martin P. 1997. Wound healing-Aiming for perfect skin regeneration. Science 276:75-81], and may be the target for the exogenous heparanase. This may also explain the increase of inflammatory cells recruited to granulation tissue observed after heparanase treatment.

Expression of heparanase gene and protein correlated with the metastatic potential of several human and mouse cell lines such as breast, bladder, prostate, melanoma and T-lymphoma [Vlodavsky I., Y. Friedman, M. Elkin, H. Aingorn, R. Atzmon, R. Ishai-Michaeli, M. Bitan, O. Pappo, T. Peretz, I. Michal, L. Spector and I. Pecker. 1999. Mammalian heparanase: Gene cloning, expression and function in tumor progression and metastasis. Nature Med. 5: 793-802]. Similarly, heparanase activity was also correlated with extravasation of immune cells during normal and chronic inflammation and with angiogenesis. Here evidence is provided, for the first time, for a direct role for heparanase in the course of wound healing and, moreover, in the regulation of sprouting angiogenesis.

A few potential clinical benefits for heparanase come to mind.

1. Heparanase may be used as a therapeutic for a wide variety of wounds under pathological conditions. These include diabetic and pressure ulcers, burns and incisional wounds, and may expand further to tissue damage caused by ischemia, mainly in the context of heart and kidney diseases. Moreover, accelerated healing may contribute to the aesthetically appearance of the wounds, implicating a potential cosmetic benefit.

2. Heparanase may be considered as an infection-inhibiting reagent. This is based upon the observation that heparanase expression is restricted to the outer most layer of the skin (stratum corneum) and the ability of various pathogenic bacteria, viruses and protozoa to bind glycosaminoglycan-based receptors on host cells, initiating infection. The combination of accelerated wound healing with inhibition of infection may provide even more potent reagent.

3. The intimate involvement in angiogenesis and the ability of heparanase to induce blood vessels formation, shown here directly for the first time, may have important clinical implication. Tumor growth is angiogenic-dependent and inhibition of blood vessel formation is sought as a cancer therapeutic. Other clinical situations critically suffer from severe tissue damage and induction of angiogenesis is believed to significantly improve tissue function. The most common and important example is ischemic heart damage, affecting millions of people every year.

4. Cutaneous wounds often cause anatomical and/or functional damage to peripheral sensory neurons widely distributed in the skin, and nerve growth factor (NGF) may be essential to regenerate the injured neurons. Neurotropic activity of NGF has been shown to be potentiating by heparin (Neufeld et al., 1987, Heparin modulation of the neurotropic effects of acidic and basic fibroblast growth factors and nerve growth factor on PC12 cells. J Cell Physiol. April 1987;131(1):131-40.) and heparan sulfate (Damon et al., 1988, Sulfated glycosaminoglycans modify growth factorinduced neurite outgrowth in PC12 cells. J Cell Physiol May 1988;135(2):293-300). Thus, heparanase activity may increase the availability of a variety of growth factors, including NGF and to support neuronal recovery.

5. As shown herein, the increase in granulation tissue cellularity is due, in part, to an increase in cell proliferation. However, a large cell population which is PCNA-negative also appears and is most likely composed of inflammatory cells. Thus, heparanase treatment may enhance the recruitment of inflammatory cells to specific sites. On the other hand, heparanase-inhibitors may prevent or reduce inflammation under several pathological conditions, including chronic and acute inflammation.

6. Heparanase expression in the skin tissue correlated with terminal cellular differentiation and keratinocytes apoptosis, while proliferating epidermal cells, stained positively for PCNA, expressed only very low levels of heparanase. Interestingly, heparanase was found to be localized to the nucleus of hair follicle cells, while cytoplasmic staining was observed in keratinocytes. This may suggest a new potential function for heparanase, other than the traditional ones. More specifically, heparanase localization to the nucleus may be involve in the regulation of gene expression, most likely due to heparanase-associating factors, and cell fate.

Heparan sulfate is found throughout the epidermis [Tanuni R H et al; Histochem. 1987, 87:243-50], but its function is unknown. The role of heparanase in normal, aging and pathological conditions of the skin is also not known, in part due to the lack of specific anti-heparanase antibodies and a purified enzyme. A few reports that describe altered HS metabolism, due to both quantitative and qualitative changes, may suggest a role for the heparanase enzyme, or its inhibitors, in the treatment of various skin conditions: It was found that cells which had aged in vivo, or in vitro, had an increased proportion of HSPG [Kent WM et al; Mech Aging Dev. 1986, 33:115-37]. It was also found that HS and blood vessels staining were increased in wounds of old animals at late time points, but the dermal organization was similar to that of normal skin. In contrast, young animals developed abnormal, dense scars. Intriguingly, some of the age-related changes in scar quality and inflammatory cell profile were similar to those seen in fetal wound healing [Ashcroft GS et al; J Invest Dermatol. 1997, 108:430-7]. Another paper showed that under the influence of chronic UVB radiation animals exhibited a marked increase in the synthesis of HS [Margelin D et al; Photochem Photobiol. 1993, 58:211-8]. HSPGs distribution changes during the differentiation stages of hair growth cycle, and they have an inductive effect on hair growth, both when injected and in diseases that result in accumulation of polysaccharides in the dermis [Westgate G et al; J Invet Dermatol. 1991, 96:191-5]. In addition to putative roles of HS in basement membrane assembly, and cell-matrix interactions, growth factor sequestration may be important for the hair follicle [Couchman J R et al; J Invest Dermatol. 1995, 104:40S]. Administration of exogenous bFGF has prolonged and marked effects on mouse hair follicle development and cycling [du Cros D L; Dev Biol. 1993, 156:444-53]. The heparin binding keratinocyte growth factors human derived keratinocyte autocrine factor (KAF) and amphiregulin (AR) can be negatively regulated by heparin [Cook P W et al; Mol Cell Biol. 1991, 11:2547-57].

As described herein in the Examples section that follows, using an anti-heparanase monoclonal antibody (HP-92) cultures of HaCat keratinocytes cell line were immunostained. These cells exhibited significant heparanase staining in their cytoplasm. Moreover, intact cells, as well as an extract of these cells, exhibited heparanase activity when assayed in an ECM-assay. Immuno-staining of normal skin tissues resulted in the intense staining of heparanase both in the dermis and epidermis.

The following described potential applications of heparanase and/or heparanase inhibitors in skin and hair care:

Heparanase treatment may improve the appearance of the skin damaged by UV irradiation and aging. Removal of excess heparan sulfate following UV light may restore natural skin (a process termed “biochemical peeling”).

Heparanase treatment may aid in skin healing via its mitogenic and angiogenic properties.

Heparanase treatment may have regenerative properties for hair growth via mitogenesis and angiogenesis.

Heparanase inhibitors may prevent minor skin inflammations, irritations and allergies via inhibition of the inflammatory immune cell response.

Heparanase inhibitors may increase levels of heparan sulfate and by that affect hair growth, skin resiliency, etc.

To facilitate understanding of the invention set forth in this disclosure, a number of terms are defined below.

The term “wound” refers broadly to injuries to the skin and subcutaneous tissue initiated in any one of a variety of ways (e.g., pressure sores from extended bed rest, wounds induced by trauma, cuts, ulcers, bums and the like) and with varying characteristics. Wounds are typically classified into one of four grades depending on the depth of the wound: (i) Grade I: wounds limited to the epithelium; (ii) Grade II: wounds extending into the dermis; (iii) Grade III: wounds extending into the subcutaneous tissue; and (iv) Grade IV (or full-thickness wounds): wounds wherein bones are exposed (e.g., a bony pressure point such as the greater trochanter or the sacrum). The term “partial thickness wound” refers to wounds that encompass Grades I-III; examples of partial thickness wounds include burn wounds, pressure sores, venous stasis ulcers, and diabetic ulcers. The term “deep wound” is meant to include both Grade III and Grade IV wounds.

The term “healing” in respect to a wound refers to a process to repair a wound as by scar formation.

The phrase “inducing or accelerating a healing process of a wound” refers to either the induction of the formation of granulation tissue of wound contraction and/or the induction of epithelialization (i.e., the generation of new cells in the epithelium). Wound healing is conveniently measured by decreasing wound area.

The present invention contemplates treating all wound types, including deep wounds and chronic wounds.

The term “chronic wound” refers a wound that has not healed within 30 days.

The phrase “transforming cells” refers to a transient or permanent alteration of a cell's nucleic acid content by the incorporation of exogenous nucleic acid which either integrates into the cell genome and genetically modifies the cell or remains unintegrated.

The phrase “cis-acting element” is used herein to describe a genetic element that is located upstream of a coding sequence and controls the expression of a protein from the coding sequence. Such elements include promoters and enhancers.

The term “angiogenesis” is used herein to described the process of blood vessels formation.

Wound healing and angiogenesis according to the present invention are induced and/or accelerated by the presence of heparanase. As is demonstrated herein, heparanase, by degrading HS releases and/or activates a plurality of factors which evidently induce and/or accelerate wound healing and angiogenesis, wherein wound healing is induced or accelerated by induced or accelerated angiogenesis and inflammation, whereas angiogenesis itself is induced by release of angiogenic factors from the ECM.

The phrase “heparanase coated cells” refers to cells to which natural or recombinant, active or activatable (proenzyme) heparanase was externally adhered ex vivo. Such cells can form a part of a tissue soaked in a heparanase containing solution.

Thus, according to one aspect of the present invention there is provided a method of inducing or accelerating a healing process of a wound and/or angiogenesis. The method according to this aspect of the invention is effected by administering a therapeutically effective amount of heparanase, so as to induce or accelerate the healing process of the wound and/or angiogenesis.

According to another aspect of the present invention there is provided a pharmaceutical composition for inducing or accelerating a healing process of a wound and/or angiogenesis. The pharmaceutical composition comprising, as an active ingredient, heparanase and a pharmaceutically acceptable carrier.

According to yet another aspect of the present invention there is provided a method of inducing or accelerating a healing process of a wound and/or angiogenesis. The method according to this aspect of the invention is effected by implanting a therapeutically effective amount of heparanase expressing or secreting cells, or heparanase coated cells, so as to induce or accelerate the healing process of the wound and/or angiogenesis.

According to still another aspect of the present invention there is provided a pharmaceutical composition for inducing or accelerating a healing process of a wound and/or angiogenesis. The pharmaceutical composition according to this aspect of the invention comprising, as an active ingredient, heparanase expressing or secreting cells, or heparanase coated cells, and a pharmaceutically acceptable carrier.

According to an additional aspect of the present invention there is provided a method of inducing or accelerating a healing process of a wound and/or angiogenesis. The method according to this aspect of the invention is effected by transforming cells in vivo to produce and secrete heparanase, so as to induce or accelerate the healing process of the wound and/or angiogenesis.

According to yet an additional aspect of the present invention there is provided a pharmaceutical composition for inducing or accelerating a healing process of a wound and/or angiogenesis. The pharmaceutical composition according to this aspect of the invention comprising, as an active ingredient, a nucleic acid construct being designed for transforming cells in vivo to produce and secrete heparanase, and a pharmaceutically acceptable carrier.

Thus, wound healing and angiogenesis according to the present invention are induced and/or accelerated by heparanase.

One way is the direct administration of heparanase. Heparanase can be purified from natural sources or produced by recombinant technology.

In an alternative embodiment, cells expressing or secreting heparanase are implanted in vivo, so as to induce or accelerate the healing process of a wound or induce angiogenesis. Such heparanase producing cells may be cells naturally producing heparanase, or alternatively, such cells are transformed to produce and secrete heparanase. The cells can be transformed by a cis-acting element sequence, such as a strong and constitutive or inducible promoter integrated upstream to an endogenous heparanase gene of the cells, by way of gene knock-in, and produce and secrete natural heparanase. It will be appreciated that the still alternatively, the cells can be transformed by a recombinant heparanase gene to produce and secrete recombinant heparanase.

Advantageously, the heparanase expressing or secreting cells are capable of forming secretory granules, so as to secrete heparanase produced thereby. The heparanase expressing or secreting cells can be endocrine cells. They can be of a human source or of a histocompatibility humanized animal source. Most preferably, the heparanase expressing or secreting cells, either transformed or not, are of an autologous source. The heparanase produced by the heparanase expressing or secreting cells is preferably human heparanase or has the amino acid sequence of human heparanase. The heparanase expressing or secreting cells can be fibroblasts, epithelial cells, keratinocytes or cells present in a full thickness skin, provided that a transformation as described herein is employed so as to render such cells to produce and secrete heparanase. Cells or tissue such as full thickness skin implant or transplant can be coated with heparanase. Thus the cells of the present invention can be isolated cells or cells embedded in a tissue implant or transplant.

In still an alternative embodiment cells are transformed in vivo to produce and secrete heparanase, so as to induce or accelerate the healing process of a wound and/or angiogenesis.

Any one of a plurality of transformation approaches described above, e.g., transformation with a construct encoding heparanase, or transformation with a construct harboring a cis-acting element for activation of endogenous heparanase production and secretion, can be employed in context of this embodiment of the present invention.

In some aspects the present invention utilizes in vivo and ex vivo (cellular) gene therapy techniques which involve cell transformation and gene knock-in type transformation. Gene therapy as used herein refers to the transfer of genetic material (e.g., DNA or RNA) of interest into a host to treat or prevent a genetic or acquired disease or condition or phenotype. The genetic material of interest encodes a product (e.g., a protein, polypeptide, peptide, functional RNA, antisense RNA) whose production in vivo is desired. For example, the genetic material of interest can encode a hormone, receptor, enzyme, polypeptide or peptide of therapeutic value. For review see, in general, the text “Gene Therapy” (Advanced in Pharmacology 40, Academic Press, 1997).

Two basic approaches to gene therapy have evolved (1) ex vivo; and (ii) in vivo gene therapy. In ex vivo gene therapy cells are removed from a patient or are derived from another source, and while being cultured are treated in vitro. Generally, a functional replacement gene is introduced into the cell via an appropriate gene delivery vehicle/method (transfection, transduction, homologous recombination, etc.) and an expression system as needed and then the modified cells are expanded in culture and returned to lo the host/patient. These genetically reimplanted cells have been shown to express the transfected genetic material in situ.

In in vivo gene therapy, target cells are not removed from the subject rather the genetic material to be transferred is introduced into the cells of the recipient organism in situ, that is within the recipient. In an alternative embodiment, if the host gene is defective, the gene is repaired in situ [Culver, 1998. (Abstract) Antisense DNA & RNA based therapeutics, February 1998, Coronado, Calif.]. These genetically altered cells have been shown to express the transfected genetic material in situ.

The gene expression vehicle is capable of delivery/transfer of heterologous nucleic acid into a host cell. The expression vehicle may include elements to control targeting, expression and transcription of the nucleic acid in a cell selective manner as is known in the art. It should be noted that often the 5′UTR and/or 3′UTR of the gene may be replaced by the 5′UTR and/or 3′UTR of the expression vehicle. Therefore, as used herein the expression vehicle may, as needed, not include the 5′UTR and/or 3′UTR of the actual gene to be transferred and only include the specific amino acid coding region.

The expression vehicle can include a promoter for controlling transcription of the heterologous material and can be either a constitutive or inducible promoter to allow selective transcription. Enhancers that may be required to obtain necessary transcription levels can optionally be included. Enhancers are generally any nontranslated DNA sequence which works contiguously with the coding sequence (in cis) to change the basal transcription level dictated by the promoter. The expression vehicle can also include a selection gene as described herein below.

Vectors can be introduced into cells or tissues by any one of a variety of known methods within the art. Such methods can be found generally described in Sambrook et al., Molecular Cloning: A Laboratory Manual, Cold Springs Harbor Laboratory, New York 1989, 1992, in Ausubel et al., Current Protocols in Molecular Biology, John Wiley and Sons, Baltimore, Md. 1989, Chang et al., Somatic Gene Therapy, CRC Press, Ann Arbor, Mich. 1995, Vega et al., Gene Targeting, CRC Press, Ann Arbor Mich. (995), Vectors: A Survey of Molecular Cloning Vectors and Their Uses, Butterworths, Boston Mass. 1988 and Gilboa et al., Biotechniques 4 (6): 504-512, 1986, and include, for example, stable or transient transfection, lipofection, electroporation and infection with recombinant viral vectors. In addition, see U.S. Pat. No. 4,866,042 for vectors involving the central nervous system and also U.S. Pat. Nos. 5,464,764 and 5,487,992 for positive-negative selection methods.

Introduction of nucleic acids by infection offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

A specific example of DNA viral vector introducing and expressing recombination sequences is the adenovirus-derived vector Adenop53TK. This vector expresses a herpes virus thymidine kinase (TK) gene for either positive or negative selection and an expression cassette for desired recombinant sequences. This vector can be used to infect cells that have an adenovirus receptor which includes most tissues of epithelial origin as well as others. This vector as well as others that exhibit similar desired functions can be used to treat a mixed population of cells and can include, for example, in vitro or ex vivo culture of cells, a tissue or a human subject.

Features that limit expression to particular cell types can also be included. Such features include, for example, promoter and regulatory elements that are specific for the desired cell type.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result-is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

As described above, viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. The vector to be used in the methods and compositions of the invention will depend on desired cell type to be targeted and will be known to those skilled in the art.

Retroviral vectors can be constructed to function either as infectious particles or to undergo only a single initial round of infection. In the former case, the genome of the virus is modified so that it maintains all the necessary genes, regulatory sequences and packaging signals to synthesize new viral proteins and RNA. Once these molecules are synthesized, the host cell packages the RNA into new viral particles which are capable of undergoing further rounds of infection. The vector's genome is also engineered to encode and express the desired recombinant gene. In the case of non-infectious viral vectors, the vector genome is usually mutated to destroy the viral packaging signal that is required to encapsulate the RNA into viral particles. Without such a signal, any particles that are formed will not contain a genome and therefore cannot proceed through subsequent rounds of infection. The specific type of vector will depend upon the intended application. The actual vectors are also known and readily available within the art or can be constructed by one skilled in the art using well-known methodology.

The recombinant vector can be administered in several ways. If viral vectors are used, for example, the procedure can take advantage of their target specificity and consequently, do not have to be administered locally at the diseased site. However, local administration can provide a quicker and more effective treatment.

Procedures for in vivo and ex vivo cell transformation including homologous recombination employed in knock-in procedures are set forth in, for example, U.S. Pat. Nos. 5,487,992, 5,464,764, 5,387,742, 5,360,735, 5,347,075, 5,298,422, 5,288,846, 5,221,778, 5,175,385, 5,175,384, 5,175,383, 4,736,866 as well as Burke and Olson, Methods in Enzymology, 194:251-270 1991); Capecchi, Science 244:1288-1292 1989); Davies et al., Nucleic Acids Research, 20 (11) 2693-2698 1992); Dickinson et al., Human Molecular Genetics, 2(8): 1299-1302 1993); Duff and Lincoln, “Insertion of a pathogenic mutation into a yeast artificial chromosome containing the human APP gene and expression in ES cells”, Research Advances in Alzheimer's Disease and Related Disorders, 1995; Huxley et al., Genomics, 9:742-750 1991); Jakobovits et al., Nature, 362:255-261 1993); Lamb et al., Nature Genetics, 5: 22-29 1993); Pearson and Choi, Proc. Natl. Acad. Sci. USA 1993). 90:10578-82; Rothstein, Methods in Enzymology, 194:281-301 1991); Schedl et al., Nature, 362: 258-261 1993); Strauss et al., Science, 259:1904-1907 1993). Further, patent applications WO 94/23049, WO93/14200, WO 94/06908, WO 94/28123 also provide information.

Thus, transformations according to the present invention can employ naked DNA or viral vectors to introduce a sequence of interest into cells. Viral vectors are developed by modification of the viral genome in the form of replicative defective viruses. The most widely used viral vectors are the retroviruses and adenoviruses, which are used for experimental as well as gene therapy purposes [Kuroki, T., Kashiwagi, M., Ishino, K., Huh, N., and Ohba, M. Adenovirus-mediated gene transfer to keratinocytes—a review. J. Investig. Dermatol. Symp. Proc., 4: 153-157, 1999]. Specifically, the high efficiency of adenovirus infection in non replicating cells, the high titer of virus and the high expression of the transduced protein makes this system highly advantageous to primary cultures compared to retroviral vectors. As adenoviruses do not integrate into the host genome and the stable viral titers can be rendered replication deficient, these viral constructs are associated with minimal risk for malignancies in human as well as animal models (Rosenfeld, M. A., Siegfried, W., Yoshimura, K., Yoneyama, K., Fukayama, M., Stier, L. E., Paakko, P. K., Gi, P., Stratford-Perricaudet, M., Jallet, J., Pavirani, A., Lecocq, J. P., and Crystal, R. G. Adenovirus-mediated transfer of a recombinant al-antitrypsin gene to the lung epithelium in vivo. Science, 252: 431-434, 1991). To date, in skin, adenovirus constructs have also been used successfully with high efficiency of infection with ex vivo and in vivo approaches [Setoguchi, Y., Jaffe, H. A., Danel, C., and Crystal, R. G. Ex Vivo and in vivo gene transfer to the skin using replication deficient recombinant adenovirus vectors. J. Invest. Dermatol., 102: 415-421, 1994; Greenhalgh, D. A., Rothnagel, J. A., and Roop, D. R. Epidermis: An attractive target tissue for gene therapy. J. Invest. Dermatol., 103: 63S-69S, 1994]. An adenovirus vector, which was developed by I. Saito and his associates [Miyake, S., Makimura, M., Kanegae, Y., Harada, S., Sato, Y., Takamori, K., Tokuda, C., and Saito, I. Efficient generation of recombinant adenoviruses using adenovirus DNA-terminal protein complex and a cosmid bearing the full-length virus genome. Proc. Natl. Acad. Sci. U.S.A., 93: 1320-1324, 1996] was used in the present study. The cosmid cassette (pAxCAwt) has nearly a full length adenovirus 5 genome but lacks E1A, E1B and E3 regions, rendering the virus replication defective. It contains a composite CAG promoter, consisting of the cytomegalovirus immediate-early enhancer, chicken β-actin promoter, and a rabbit β-globin polyadenylation signal, which strongly induces expression of inserted DNAs [Kuroki, T., Kashiwagi, M., Ishino, K., Huh, N., and Ohba, M. Adenovirus-mediated gene transfer to keratinocytes—a review. J. Investig. Dermatol. Symp. Proc., 4: 153-157, 1999; Miyake, S., Makimura, M., Kanegae, Y., Harada, S., Sato, Y., Takamori, K., Tokuda, C., and Saito, I. Efficient generation of recombinant adenoviruses using adenovirus DNA-terminal protein complex and a cosmid bearing the full-length virus genome. Proc. Natl. Acad. Sci. U.S.A., 93: 1320-1324, 1996]. A gene of interest is inserted into the cosmid cassette, which is then co-transfected into human embryonic kidney 293 cells together with adenovirus DNA terminal protein complex (TPC). In 293 cells that express E1A and E1B regions, recombination occurs between the cosmid cassette and adenovirus DNA-TPC, yielding the desired recombinant virus at an efficiency 100-fold that of conventional methods. Such high efficiency is mainly due to the use of the adenovirus DNA-TPC instead of proteinesed DNA. Furthermore, the presence of longer homologous regions increases the efficiency of the homologous recombination. Regeneration of replication competent viruses is avoided due to the presence of multiple EcoT221 sites.

The therapeutically/pharmaceutically active ingredients of the present invention can be administered per se, or in a pharmaceutical composition mixed with suitable carriers and/or excipients. Pharmaceutical compositions suitable for use in context of the present invention include those compositions in which the active ingredients are contained in an amount effective to achieve an intended therapeutic effect.

As used herein a “pharmaceutical composition” refers to a preparation of one or more of the active ingredients described herein, either protein, nucleic acids or cells, or physiologically acceptable salts or prodrugs thereof, with other chemical components such as traditional drugs, physiologically suitable carriers and excipients. The purpose of a pharmaceutical composition is to facilitate administration of a compound or cell to an organism. Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Hereinafter, the phrases “physiologically suitable carrier” and “pharmaceutically acceptable carrier” are interchangeably used and refer to a carrier or a diluent that does not cause significant irritation to an organism and does not abrogate the biological activity and properties of the administered conjugate.

Herein the term “excipient” refers to an inert substance added to a pharmaceutical composition to further facilitate processes and administration of the active ingredients. Examples, without limitation, of excipients include calcium carbonate, calcium phosphate, various sugars and types of starch, cellulose derivatives, gelatin, vegetable oils and polyethylene glycols.

Techniques for formulation and administration of active ingredients may be found in “Remington's Pharmaceutical Sciences,” Mack Publishing Co., Easton, Pa., latest edition, which is incorporated herein by reference.

While various routes for the administration of active ingredients are possible, and were previously described, for the purpose of the present invention, the topical route is preferred, and is assisted by a topical carrier. The topical carrier is one, which is generally suited for topical active ingredients administration and includes any such materials known in the art. The topical carrier is selected so as to provide the composition in the desired form, e.g., as a liquid or non-liquid carrier, lotion, cream, paste, gel, powder, ointment, solvent, liquid diluent, drops and the like, and may be comprised of a material of either naturally occurring or synthetic origin. It is essential, clearly, that the selected carrier does not adversely affect the active agent or other components of the topical formulation, and which is stable with respect to all components of the topical formulation. Examples of suitable topical carriers for use herein include water, alcohols and other nontoxic organic solvents, glycerin, mineral oil, silicone, petroleum jelly, lanolin, fatty acids, vegetable oils, parabens, waxes, and the like. Preferred formulations herein are colorless, odorless ointments, liquids, lotions, creams and gels.

Ointments are semisolid preparations, which are typically based on petrolatum or other petroleum derivatives. The specific ointment base to be used, as will be appreciated by those skilled in the art, is one that will provide for optimum active ingredients delivery, and, preferably, will provide for other desired characteristics as well, e.g., emolliency or the like. As with other carriers or vehicles, an ointment base should be inert, stable, nonirritating and nonsensitizing. As explained in Remington: The Science and Practice of Pharmacy, 19th Ed. (Easton, Pa.: Mack Publishing Co., 1995), at pages 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight; again, reference may be made to Remington: The Science and Practice of Pharmacy for further information.

Lotions are preparations to be applied to the skin surface without friction, and are typically liquid or semiliquid preparations, in which solid particles, including the active agent, are present in a water or alcohol base. Lotions are usually suspensions of solids, and may comprise a liquid oily emulsion of the oil-in-water type. Lotions are preferred formulations herein for treating large body areas, because of the ease of applying a more fluid composition. It is generally necessary that the insoluble matter in a lotion be finely divided. Lotions will typically contain suspending agents to produce better dispersions as well as active ingredients useful for localizing and holding the active agent in contact with the skin, e.g., methylcellulose, sodium carboxymethylcellulose, or the like.

Creams containing the selected active ingredients are, as known in the art, viscous liquid or semisolid emulsions, either oil-in-water or water-in-oil. Cream bases are water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation, as explained in Remington, supra, is generally a nonionic, anionic, cationic or amphoteric surfactant.

Gel formulations are preferred for application to the scalp. As will be appreciated by those working in the field of topical active ingredients formulation, gels are semisolid, suspension-type systems. Single-phase gels contain organic macromolecules distributed substantially uniformly throughout the carrier liquid, which is typically aqueous, but also, preferably, contain an alcohol and, optionally, an oil.

Carriers for nucleic acids include, but are not limited to, liposomes including targeted liposomes, nucleic acid complexing agents, viral coats and the like. However, transformation with naked nucleic acids may also be employed.

Various additives, known to those skilled in the art, may be included in the topical formulations of the invention. For example, solvents may be used to solubilize certain active ingredients substances. Other optional additives include skin permeation enhancers, opacifiers, anti-oxidants, gelling agents, thickening agents, stabilizers, and the like.

As has already been mentioned hereinabove, topical preparations for the treatment of wounds according to the present invention may contain other pharmaceutically active agents or ingredients, those traditionally used for the treatment of such wounds. These include immunosuppressants, such as cyclosporine, antimetabolites, such as methotrexate, corticosteroids, vitamin D and vitamin D analogs, vitamin A or its analogs, such etretinate, tar, coal tar, anti pruritic and keratoplastic agents, such as cade oil, keratolytic agents, such as salicylic acid, emollients, lubricants, antiseptic and disinfectants, such as the germicide dithranol (also known as anthralin) photosensitizers, such as psoralen and methoxsalen and UV irradiation. Other agents may also be added, such as antimicrobial agents, antifungal agents, antibiotics and anti-inflammatory agents. Treatment by oxygenation (high oxygen pressure) may also be co-employed.

The topical compositions of the present invention may also be delivered to the skin using conventional dermal-type patches or articles, wherein the active ingredients composition is contained within a laminated structure, that serves as a drug delivery device to be affixed to the skin. In such a structure, the active ingredients composition is contained in a layer, or “reservoir”, underlying an upper backing layer. The laminated structure may contain a single reservoir, or it may contain multiple reservoirs. In one embodiment, the reservoir comprises a polymeric matrix of a pharmaceutically acceptable contact adhesive material that serves to affix the system to the skin during active ingredients delivery. Examples of suitable skin contact adhesive materials include, but are not limited to, polyethylenes, polysiloxanes, polyisobutylenes, polyacrylates, polyurethanes, and the like. The particular polymeric adhesive selected will depend on the particular active ingredients, vehicle, etc., i.e., the adhesive must be compatible with all components of the active ingredients-containing composition. Alternatively, the active ingredients-containing reservoir and skin contact adhesive are present as separate and distinct layers, with the is adhesive underlying the reservoir which, in this case, may be either a polymeric matrix as described above, or it may be a liquid or hydrogel reservoir, or may take some other form.

The backing layer in these laminates, which serves as the upper surface of the device, functions as the primary structural element of the laminated structure and provides the device with much of its flexibility. The material selected for the backing material should be selected so that it is substantially impermeable to the active ingredients and to any other components of the active ingredients-containing composition, thus preventing loss of any components through the upper surface of the device. The backing layer may be either occlusive or nonocclusive, depending on whether it is desired that the skin become hydrated during active ingredients delivery. The backing is preferably made of a sheet or film of a preferably flexible elastomeric material. Examples of polymers that are suitable for the backing layer include polyethylene, polypropylene, and polyesters.

During storage and prior to use, the laminated structure includes a release liner. Immediately prior to use, this layer is removed from the device to expose the basal surface thereof, either the active ingredients reservoir or a separate contact adhesive layer, so that the system may be affixed to the skin. The release liner should be made from an active ingredients/vehicle impermeable material.

Such devices may be fabricated using conventional techniques, known in the art, for example by casting a fluid admixture of adhesive, active ingredients and vehicle onto the backing layer, followed by lamination of the release liner. Similarly, the adhesive mixture may be cast onto the release liner, followed by lamination of the backing layer. Alternatively, the active ingredients reservoir may be prepared in the absence of active ingredients or excipient, and then loaded by “soaking” in an active ingredients/vehicle mixture.

As with the topical formulations of the invention, the active ingredients composition contained within the active ingredients reservoirs of these laminated system may contain a number of components. In some cases, the active ingredients may be delivered “neat,” i.e., in the absence of additional liquid. In most cases, however, the active ingredients will be dissolved, dispersed or suspended in a suitable pharmaceutically acceptable vehicle, typically a solvent or gel. Other components, which may be present, include preservatives, stabilizers, surfactants, and the like.

The pharmaceutical compositions herein described may also comprise suitable solid or gel phase carriers or excipients. Examples of such carriers or excipients include, but are not limited to, calcium carbonate, calcium phosphate, various sugars, starches, cellulose derivatives, gelatin and polymers such as polyethylene glycols.

Other suitable routes of administration may, for example, include oral, rectal, transmucosal, transdermal, intestinal or parenteral delivery, including intramuscular, subcutaneous and intramedullary injections as well as intrathecal, direct intraventricular, intravenous, intraperitoneal, intranasal, or intraocular injections.

Pharmaceutical compositions for use in accordance with the present invention thus may be formulated in conventional manner using one or more pharmaceutically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological saline buffer. For transmucosal administration, penetrants are used in the formulation. Such penetrants are generally known in the art.

For oral administration, the active ingredients can be formulated readily by combining the active ingredients with pharmaceutically acceptable carriers well known in the art. Such carriers enable the active ingredients of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active ingredient doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the active ingredient and a suitable powder base such as lactose or starch.

The active ingredients described herein may be formulated for parenteral administration, e.g., by bolus injection or continues infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water, before use.

The active ingredients of the present invention may also be formulated in rectal compositions such as suppositories or retention enemas, using, e.g., conventional suppository bases such as cocoa butter or other glycerides.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Pharmaceutical compositions suitable for use in context of the present invention include compositions wherein the active ingredients are contained in an amount effective to achieve the intended purpose. More specifically, a therapeutically-effective amount means an amount of active ingredient effective to prevent, alleviate or ameliorate symptoms of disease or prolong the survival of the subject being treated.

Determination of a therapeutically effective amount is well within the capability of those skilled in the art, especially in light of the detailed disclosure provided herein.

For any active ingredient used in the methods of the invention, the therapeutically effective amount or dose can be estimated initially from activity assays in animals. For example, a dose can be formulated in animal models to achieve a circulating concentration range that includes the IC₅₀ as determined by activity assays. Such information can be used to more accurately determine useful doses in humans.

Toxicity and therapeutic efficacy of the active ingredients described herein can be determined by standard pharmaceutical procedures in experimental animals, e.g., by determining the IC₅₀ and the LD₅₀ (lethal dose causing death in 50% of the tested animals) for a subject active ingredient. The data obtained from these activity assays and animal studies can be used in formulating a range of dosage for use in human.

The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma levels of the active moiety which are sufficient to maintain the modulating effects, termed the minimal effective concentration (MEC). The MEC will vary for each preparation, but can be estimated from in vitro data; e.g., the concentration necessary to achieve 50-90% inhibition of a kinase may be ascertained using the assays described herein. Dosages necessary to achieve the MEC will depend on individual characteristics and route of administration. HPLC assays or bioassays can be used to determine plasma concentrations.

Dosage intervals can also be determined using the MEC value. Preparations should be administered using a regimen, which maintains plasma levels above the MEC for 10-90% of the time, preferable between 30-90% and most preferably 50-90%.

Depending on the severity and responsiveness of the condition to be treated, dosing can also be a single administration of a slow release composition described hereinabove, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising an active ingredient of the invention formulated in a compatible pharmaceutical carrier may also be prepared, placed in an appropriate container, and labeled for treatment of an indicated condition.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. to E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiugi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### ANGIOGENESIS

Materials and Experimental Methods

Cells

The methylcholanthrene induced non-metastatic Eb T-lymphoma cells were grown in RPMI 1640 supplemented with 10% FCS [Vlodavsky, I. et al. Mammalian heparanase: gene cloning, expression and function in tumor progression and metastasis. Nat Med 5, 793-802 (1999)]. Bovine aortic EC were cultured in DMEM (1 gram glucose/liter) supplemented with 10% calf serum [Vlodavsky, I. in Current protocols in Cell Biology, Vol. I, Suppl. I, Eds. J. S. Bonifacino, M. Dasso, J. B. Harford, J. Lippincott-Schwartz & K. M. Yamada, John Wiley & Sons, New York, New York, pp.10.4.1-10.4.14 (1999)] (Life Technologies). Bovine corneal EC were established and maintained as described [Vlodavsky, I. in Current protocols in Cell Biology, Vol. I, Suppl. I, Eds. J. S. Bonifacino, M. Dasso, J. B. Harford, J. Lippincott-Schwartz & K. M. Yamada, John Wiley & Sons, New York, N.Y., pp.10.4.1-10.4.14 (1999)]. Cells were cultured at 37° C. in 10% CO₂ humidified incubators [Vlodavsky, I. in Current protocols in Cell Biology, Vol. I, Suppl. I, Eds. J. S. Bonifacino, M. Dasso, J. B. Harford, J. Lippincott-Schwartz & K.M. Yamada, John Wiley & Sons, New York, N.Y., pp.10.4.1-10.4.14 (1999)]. Clone F32 of BaF₃ lymphoid cells, kindly provided by Dr. D. Ornitz (Department of Molecular Biology, Washington University in St. Louis), were grown in RPMl 1640 medium supplemented with 10% FCS, 10% interleukin-3 conditioned medium produced by X63-IL3 WHEI cells, L-glutamine and antibiotics [Ornitz, D. M. et al. Heparin is required for cell-free binding of basic fibroblast growth factor to a soluble receptor and for mitogenesis in whole cells. Mol Cell Biol 12, 240-247 (1992)].

Recombinant Heparanase

Recombinant heparanase was produced in stable transfected Chinese hamster ovary (CHO) cells. The entire open reading frame of heparanase was subcloned into the EcoRI-NotI sites of the mammalian expression vector pSI (Promega), which was modified to harbor a dihydrofolate reductase expression cassette. The pSIhpa expression vector was transfected into CHO cells [Vlodavsky, I. et al. Mammalian heparanase: gene cloning, expression and function in tumor progression and metastasis. Nat Med 5, 793-802 (1999)]. Recombinant heparanase was purified from CHO cell extracts using a cation exchange CM-Sepharose column (Amersham Pharmacia Biotech).

Preparation of Dishes Coated with ECM

Bovine corneal EC were cultured as described above except that 5% dextran T-40 was included in the growth medium and the cells were maintained without addition of bFGF for 12 days. The subendothelial ECM was exposed by dissolving the cell layer with PBS containing 0.5% Triton X-100 and 20 mM NH₄OH, followed by four washed in PBS [Vlodavsky, I. in Current protocols in Cell Biology, Vol. I, Suppl. I, Eds. J. S. Bonifacino, M. Dasso, J. B. Harford, J. Lippincott-Schwartz & K. M. Yamada, John Wiley & Sons, New York, N.Y., pp.10.4.1-10.4.14 (1999)]. The ECM remained intact, free of cellular debris and firmly attached to the entire area of the tissue culture dish [Vlodavsky, I. in Current protocols in Cell Biology, Vol. I, Suppl. I, Eds. J. S. Bonifacino, M. Dasso, J. B. Harford, J. Lippincott-Schwartz & K. M. Yamada, John Wiley & Sons, New York, N.Y., pp. 10.4.1-10.4.14 (1999)]. For preparation of sulfate-labeled ECM, corneal endothelial cells were cultured in the presence of Na₂[³⁵S]O₄ (Amersham) added (25 μCi/ml) one day and 5 days after seeding and the cultures were incubated with the label without medium change [Vlodavsky, I. in Current protocols in Cell Biology, Vol. I, Suppl. I, Eds. J. S. Bonifacino, M. Dasso, J. B. Harford, J. Lippincott-Schwartz & K. M. Yamada, John Wiley & Sons, New York, N.Y., pp.10.4.1-10.4.14 (1999)]. Ten to twelve days after seeding, the cell monolayer was dissolved and the ECM exposed, as described above.

Heparanase Activity

Degradation of sulfate labeled ECM by heparanase was determined as described [Vlodavsky, I. et al. Mammalian heparanase: gene cloning, expression and function in tumor progression and metastasis. Nat Med 5, 793-802 (1999); Vlodavsky, I. in Current protocols in Cell Biology, Vol. I, Suppl. I, Eds. J. S. Bonifacino, M. Dasso, J. B. Harford, J. Lippincott-Schwartz & K. M. Yamada, John Wiley & Sons, New York, N.Y., pp.10.4.1-10.4.14 (1999)]. Briefly, ECM was incubated (24 hours, 37° C., pH 6.2) with recombinant heparanase or hpa-transfected cells and sulfate labeled material released into the incubation medium was analyzed by gel filtration on a Sepharose 6B column [Vlodavsky, I. et al. Mammalian heparanase: gene cloning, expression and function in tumor progression and metastasis. Nat Med 5, 793-802 (1999); Vlodavsky, I. in Current protocols in Cell Biology, Vol. I, Suppl. I, Eds. J. S. Bonifacino, M. Dasso, J. B. Harford, J. Lippincott-Schwartz & K. M. Yamada, John Wiley & Sons, New York, N.Y., pp.10.4.1-10.4.14 (1999)]. Intact HSPGs were eluted just after the void volume (Kav<0.2, peak I) and HS degradation fragments eluted with 0.5<Kav<0.8 (peak II) [Vlodavsky, I. et al Mammalian heparanase: gene cloning, expression and function in tumor progression and metastasis. Nat Med 5, 793-802 (1999); Vlodavsky, I. in Current protocols in Cell Biology, Vol. I, Suppl. I, Eds. J. S. Bonifacino, M. Dasso, J. B. Harford, J. Lippincott-Schwartz & K. M. Yamada, John Wiley & Sons, New York, N.Y., pp.10.4.1-10.4.14 (1999)].

Release of ECM-bound bFGF

Recombinant bFGF was iodinated using chloramine T and bound to ECM as described [Vlodavsky, I. et al. Inhibition of tumor metastasis by heparanase inhibiting species of heparin. Invasion Metastasis 14, 290-302 (1994)]. Briefly, tissue culture plates coated with ECM were incubated (3 hours, 24° C.) with 0.1 ng/ml ¹²⁵I-bFGF in PBS containing 0.02% gelatin. Unbound bFGF was removed by three washes with PBS containing 0.02% gelatin. The ECM was then incubated with increasing concentrations of recombinant heparanase at 37° C. for 3 hours. The incubation media were collected and counted in a γ-counter to determine the amount of released ¹²⁵I-bFGF. The remaining ECM was incubated (3 hours, 37° C.) with IN NaOH and the solubilized radioactivity counted in a γ-counter. The percentage of released ¹²⁵I-bFGF was calculated from the total ECM-associated radioactivity [Vlodavsky, I. et al. Inhibition of tumor metastasis by heparanase inhibiting species of heparin. Invasion Metastasis 14, 290302 (1994)].

Release of Endogenous bFGF from ECM

ECM coated 35 mm dishes were incubated (24° C., 4 hours) with either 1 ml heparanase reaction mixture (150 mM NaCl, 50 mM buffer phosphate-citrate, pH 6.2, 0.2% bovine serum albumin) or reaction buffer containing 0.5 μg/ml recombinant heparanase. ELISA (Quantikine H S human FGF basic, R&D systems) tested aliquots of the incubation medium for bFGF content. Each sample was tested in triplicates and the variation between different determinations did not exceed ±7% of the mean.

Effect of HS fragments Released by Heparanase from Cell Surfaces and ECMon BaF3 Cellproliferation

Vascular EC and intact subendothelial ECM were incubated (4 hours, 37° C.) with 1 μg/ml heparanase (P50). Increasing amounts of the incubation medium containing the released HS degradation fragments were then added to BaF3 cells (2×10⁴ cells/well; 96 well plate) in the presence of 5 ng/ml bFGF. Forty-eight hours later, ³H-thymidine (1 μCi/well) (Amersham Pharmacia Biotech) was added for 6 hours, followed by cell harvesting and measurement of ³H-thymidine incorporation [Miao, H. Q., Omitz, D. M., Aingorn, E., Ben-Sasson, S. A. & Vlodavsky, I. Modulation of fibroblast growth factor-2 receptor binding, dimerization, signaling, and angiogenic activity by a synthetic heparin-mimicking polyanionic compound. J Clin Invest 99, 1565-1575 (1997); Omitz, D. M. et al Heparin is required for cell-free binding of basic fibroblast growth factor to a soluble receptor and for mitogenesis in whole cells. Mol Cell Biol 12, 240-247 (1992)].

RNA Isolation and RT-PCR Reaction

RNA from human endothelial cells was isolated and 500 ng total RNA was subjected to reverse transcription. The resulting single stranded cDNA was amplified by PCR using human specific oligonucleotide primers as described [Vlodavsky, I. et al Mammalian heparanase: gene cloning, expression and function in tumor progression and metastasis. Nat Med 5, 793-802 (1999)]. Ten μl aliquots of the amplification products were separated on a 1.5% agarose gel and visualized by ethidium bromide staining [Vlodavsky, I. et al Mammalian heparanase: gene cloning, expression and function in tumor progression and metastasis. Nat Med 5, 793-802 (1999)].

Immunohistochemistry: Immunohistochemistry was performed as described before with minor modifications [Vlodavsky, I. et al. Mammalian heparanase: gene cloning, expression and function in tumor progression and metastasis. Nat Med 5, 793-802 (1999)]. Briefly, 5 μm sections were deparaffinized and rehydrated. Tissue was then denatured for 3 minutes in a microwave oven in citrate buffer (0.01 M, pH 6.0). Blocking steps included successive incubations in 0.2% glycine, 3% H₂O₂ in methanol and 5% goat serum. Sections were incubated with a monoclonal (mAb 92.4) anti-human heparanase antibody diluted 1:3 in PBS, or with DMEM supplemented with 10% horse serum as control, diluted as above, followed by incubation with HRP conjugated goat anti-mouse IgG+IgM antibody (Jackson). mAb 92.4 is directed against the N-terminus region of the 50 kDa enzyme. The preparation and specificity of this mAb were previously described and demonstrated [Vlodavsky, I. et al. Mammalian heparanase: gene cloning, expression and function in tumor progression and metastasis. Nat Med 5, 793-802 (1999)]. Color was developed using Zymed AEC substrate kit (Zymed) for 10 minutes, followed by counter stain with Mayer's hematoxylin [Vlodavsky, I. et al Mammalian heparanase: gene cloning, expression and function in tumor progression and metastasis. Nat Med 5, 793-802 (1999)].

Matrigel Plug Assay

Matrigel plug assay was performed as previously described [Passaniti, A. et al. A simple, quantitative method for assessing angiogenesis and antiangiogenic agents using reconstituted basement membrane, heparin, and fibroblast growth factor. Lab Invest 67, 519-528 (1992)]. Six week old male BALB/c mice (n=5) were injected subcutaneously at the lateral abdominal area with 0.4 ml of Matrigel (kindly provided by Dr. H. Kleinmann, NIDR, NIH, Bethesda, Md.) premixed on ice with 2×10⁶ hpa transfected Eb murine lymphoma cells highly expressing and secreting a recombinant heparanase [Vlodavsky, I. et al. Mammalian heparanase: gene cloning, expression and function in tumor progression and metastasis. Nat Med 5, 793-802 (1999)]. Control mice were injected with Matrigel mixed with mock-transfected Eb cells, lacking heparanase. Matrigel plugs were removed 7 days post implantation, photographed and transferred to tubes containing 0.4 ml DDW. Plugs were homogenized with a Politron homogenizer until complete disintegration. The debris was centrifuged and the hemoglobin containing supernatant was collected. Hemoglobin content was determined using Drabkin reagent (Sigma) and quantitated against a standard curve of plasma hemoglobin.

Experimental Results

Expression of heparanase by vascular EC

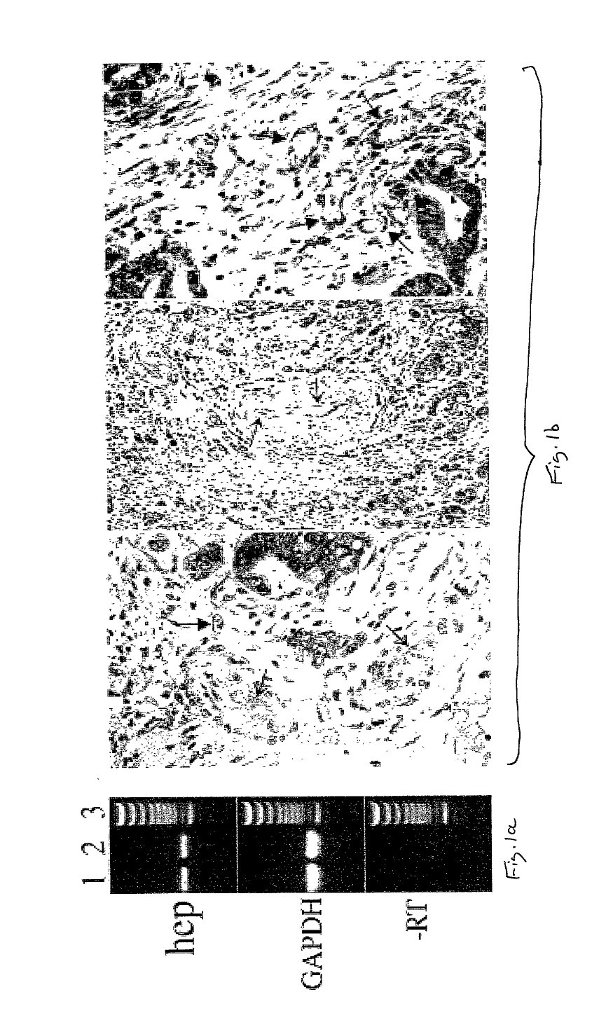

Previously, it has been suggested that stimulated EC secrete heparanase-like activity [Godder, K. et al. Heparanase activity in cultured endothelial cells. J Cell Physiol 148, 274-280 (1991); Pillarisetti, S. et al. Endothelial cell heparanase modulation of lipoprotein lipase activity. Evidence that heparan sulfate oligosaccharide is an extracellular chaperone. J Biol Chem 272, 15753-15759 (1997)]. Using RT-PCR, it was unequivocally demonstrated, for the first time, that the heparanase gene is expressed by proliferating human ECs. Both cultured human umbilical vein EC (HUVEC) and human bone marrow EC (TrHBMEC) [Schweitzer, K. M. et al. Characterization of a newly established human bone marrow endothelial cell line: distinct adhesive properties for hematopoietic progenitors compared with human umbilical vein endothelial cells. Lab Invest 76, 25-36 (1997)] expressed the heparanase gene, as reflected by the 564-bp PCR product (FIG. 1a).

Expression of Heparanase in ECs in Blood Vessels

Paraffin embedded sections from patients with primary colon adenocarcinoma were subjected to immunohistochemical staining with monoclonal anti-heparanase antibodies. An interesting pattern of staining was noted in EC in blood vessels of different maturation stages. The heparanase protein is preferentially expressed in sprouting capillaries (FIG. 1b, left and right, arrows) whereas the endothelium of mature quiescent vessels showed no detectable levels of heparanase (FIG. 1b, left and middle, concave arrows). A similar expression pattern was observed in human mammary and pancreatic carcinomas. This result suggests a significant role of endothelial heparanase in enabling EC to traverse BM and ECM barriers during sprouting angiogenesis. As previously reported [Vlodavsky, I. et al. Mammalian heparanase: gene cloning, expression and function in tumor progression and metastasis. Nat Med 5, 793-802 (1999)] and also demonstrated in FIG. 1b, the neoplastic colonic mucosa exhibited an intense heparanase staining, as opposed to no expression of heparanase in normal colon epithelium [Vlodavsky, I. et al. Mammalian heparanase: gene cloning, expression and function in tumor progression and metastasis. Nat Med 5, 793-802 (1999)]. Carcinoma cells can therefore be regarded as the main source of heparanase in the tumor microenvironment. Moreover, at a later stage of tumor progression, heparanase was also found in the tumor stroma.

Release of ECM Bound ¹²⁵I-bFGF by Heparanase

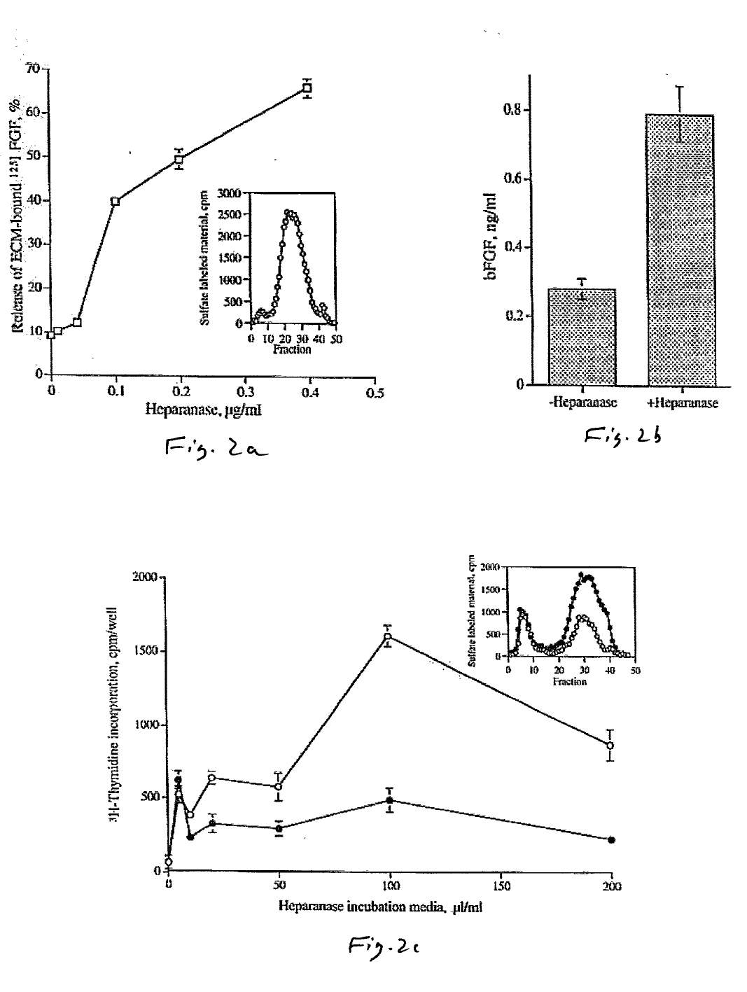

Naturally produced subendothelial ECM was preincubated with ¹²⁵I-bFGF, washed free of the unbound bFGF and incubated (3 hours, 37° C.) with the 50 kDa active form of the recombinant heparanase enzyme. As demonstrated in FIG. 2a, degradation of HS in the ECM, reflected by release of sulfate labeled HS degradation fragments (inset), resulted in release of as much as 70% of the ECM-bound ¹²⁵I-bFGF. Alternatively, the enzyme was added to native ECM that was not preincubated with ¹²⁵I-bFGF. Aliquots of the incubation medium were then tested for the presence of bFGF, using a quantitative ELISA for bFGF. Nearly 0.8 ng endogenous bFGF were released from ECM coating the surface area of a 35 mm culture dish (FIG. 2b). The released bFGF stimulated 5-8 fold the proliferation of 3T3 fibroblasts and bovine aortic EC. These results clearly indicate that heparanase releases active bFGF sequestered as a complex with HS in the ECM. Both tumor and endothelial heparanase may hence elicit an indirect angiogenic response by means of releasing active HS-FGF complexes from storage in the ECM and tumor microenvironment.

Release of ECM Bound bFGF by Heparanase—bFGF Cellular Response Assay

The ability of heparanase cleaved HS degradation fragments to promote the mitogenic activity of bFGF was investigated using a cytokinedependent lymphoid cell line (BaF3, clone 32) engineered to express FGF receptor 1 (FGFR1) [Miao, H. Q., Omitz, D. M., Aingorn, E., Ben-Sasson, S. A. & Vlodavsky, I. Modulation of fibroblast growth factor-2 receptor binding, dimerization, signaling, and angiogenic activity by a synthetic heparin-mimicking polyanionic compound. J Clin Invest 99, 1565-1575 (1997); Ornitz, D. M. et al. Heparin is required for cell-free binding of basic fibroblast growth factor to a soluble receptor and for mitogenesis in whole cells. Mol Cell Biol 12, 240-247 (1992)]. These cells lack cell surface HS and respond to bFGF only in the presence of exogenously added species of heparin or HS [Miao, H. Q., Ornitz, D. M., Aingorn, E., Ben-Sasson, S. A. & Vlodavsky, I. Modulation of fibroblast growth factor-2 receptor binding, dimerization, signaling, and angiogenic activity by a synthetic heparin mimicking polyanionic compound. J Clin Invest 99, 1565-1575 (1997); Ornitz, D. M. et al. Hepariri is required for cell-free binding of basic fibroblast growth factor to a soluble receptor and for mitogenesis in whole cells. Mol Cell Biol 12, 240-247 (1992)]. Both native ECM and confluent vascular EC monolayer were first treated with the recombinant 50 kDa heparanase enzyme. Aliquots of the incubation media were then added to BaF3 cells and tested for their ability to promote ³H-thymidine incorporation in response to bFGF. As expected, BaF3 cells exposed to either bFGF or heparanase alone exhibited almost no incorporation of ³H-thymidine. A marked stimulation (about 40 fold) of DNA synthesis was obtained in the presence of HS degradation fragments released by heparanase from EC surfaces (FIG. 2c). Interestingly, HS fragments released by heparanase from the subendothelial ECM exerted a much smaller effect (FIG. 2c). These results indicate that the heparanase enzyme potentiates the mitogenic activity of bFGF and possibly other heparin-binding angiogenic growth factors, through release of HS degradation fragments that promote bFGF-receptor binding and activation. The observed difference in biological activity between cell surface- and ECM-derived HS fragments indicates that the primary role of HS in the ECM is to sequester, protect and stabilize heparin-binding growth factors, while the cell surface HS plays a more active role in promoting the mitogenic and angiogenic activities of the growth factor by means of stimulating receptor binding, dimerization and activation. This concept is supported by the recently reported preferential ability of cell surface- vs. ECM-HSPG to mediate the assembly of bFGF-receptor signaling complex [Chang, Z., Meyer, K., Rapraeger, A. C. & Friedl, A. Differential ability of heparan sulfate proteoglycans to assemble the fibroblast growth factor receptor complex in situ. FASEB J 14, 137-144 (2000)]. The biochemical nature of (e.g., size, sequence) of oligosaccharides released by heparanase from cells vs. ECM is being characterized.

Induction of Angiogenesis into a Matrigel plug in Vivo

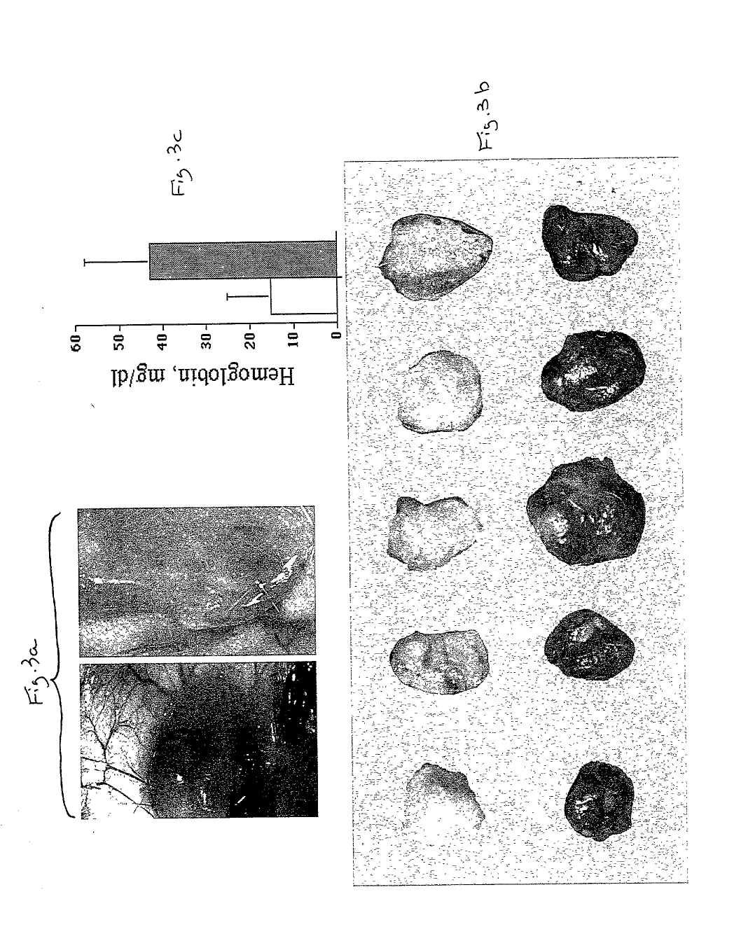

The Matrigel plug assay [Passaniti, A. et al. A simple, quantitative method for assessing angiogenesis and antiangiogenic agents using reconstituted basement membrane, heparin, and fibroblast growth factor. Lab Invest 67, 519-528 (1992)] was applied to investigate whether the heparanase enzyme can elicit an angiogenic response in vivo. For this purpose, stable heparanase transfected Eb lymphoma cells [Vlodavsky, I. et al. Mammalian heparanase: gene cloning, expression and function in tumor progression and metastasis. Nat Med 5, 793-802 (1999)] were mixed at 4° C. with Matrigel (reconstituted BM preparation extracted from EHS mouse sarcoma) and injected subcutaneously into BALB/c mice. Similarly treated mock-transfected Eb cells expressing no heparanase activity served as a control [Vlodavsky, I. et al. Mammalian heparanase: gene cloning, expression and function in tumor progression and metastasis. Nat Med 5, 793-802 (1999)]. Upon injection, the liquid Matrigel rapidly forms a solid gel plug that serves as a supporting medium for the lymphoma cells. Its major components, similar to intact BM, are laminin, collagen type IV and HSPGs. Matrigel also contains bFGF and other growth factors that are naturally found in BM and ECM [Vukicevic, S. et al. Identification of multiple active growth factors in basement membrane Matrigel suggests caution in inhibition of cellular activity related to extracellular matrix components. Exp Cell Res 202, 1-8 (1992)]. Hence, the Matrigel in this experimental system serves not merely as an inert vehicle for the enzyme producing cells, but rather maintains the natural interactions existing between tumor cells and the surrounding ECM, providing, among other effects, a source of ECM-sequestered bFGF. As shown in FIG. 3, a pronounced angiogenic response was induced by Matrigel embedded Eb cells over expressing the heparanase enzyme, as compared to little or no neovascularization exerted by mock transfected Eb cells expressing no heparanase activity. The angiogenic response was reflected by a network of capillary blood vessels attracted toward the Matrigel plug containing heparanase transfected (FIG. 3a, left) vs. control mock transfected (FIG. 3a, right) Eb cells, and by a large amount of blood and vessels seen in the isolated Matrigel plugs excised from each of the mice (FIG. 3b, bottom vs. top, respectively). This difference was highly significant, as also demonstrated by measurements of the hemoglobin content of Matrigel plugs removed from each mouse of the respective groups (FIG. 3c).

### WOUND HEALING

Materials and Experimental Methods

Wound formation and treatment

Full-thickness wound were created with a 8 mm punch at the back of 10 anesthetized Balb C male mice skin. Purified 50 kDa active heparanase enzyme was applied topically twice a day at 1 μg/wound (about 2 ng/mm²) for 4 days, and once a day for the next 3 days. Wound closure was monitored after seven days with a fine digital caliber. Average wound areas were statistically analyzed by the two-sample t-test assuming equal variances.

Histological Examination of Heparanase Treated Wounds

For histological examination, wound areas including the underlying granulation tissue, were removed and formalin-fixed paraffin-embedded sections were stained with hematoxylin-eosin. Immunohistochemistry was performed as previously described [Ilan N., S. Mahooti, D. L. Rimm and Joseph A. Madri. 1999. PECAM-1 (CD31) functions as a reservoir for and a modulator of tyrosine-phosphorylated beta-catenin. J. Cell Sci. 112: 3005-3014]. Briefly, sections were subjected to antigen retrieval, blocked with 10% normal horse serum and incubated with anti-PECAM-1, anti-PCNA (Santa Cruz) and affinity purified anti-heparanase polyclonal antibodies over night at 4° C. Sections were then washed three times with PBS and staining was visualized by the Vectastain ABC kit and DAB substrate (Vector).

Experimental Results

Wound closure

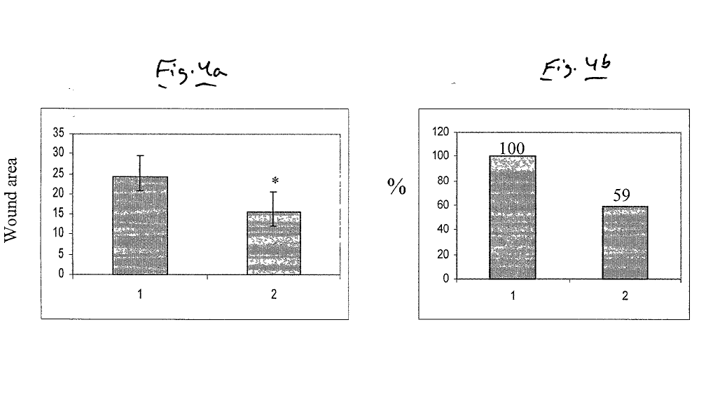

In order to directly study the effect of heparanase on the complex of events resulting in wound healing, 1 μg (in 20 μl saline) active heparanase was applied topically onto full-thickness wounds. This reflects a ten-fold less protein compared with a previous study focusing on the role of nerve growth factor (NGF) in wound healing [Hiroshi M., H. Koyama, H. Sato, J. Sawada, A. Itakura, A. Tanaka, M. Matsumoto, K. Konno, H. Ushio and K. Matsuda. 1998. Role of nerve growth factor in cutaneous wound healing: Accelerating effect in normal and healing-impaired diabetic mice. J. Exp. Med. 187: 297-303]. Careful evaluation of wound areas revealed a significant improvement of wound closure upon heparanase treatment (FIGS. 4a-b). Thus, while average wound area was 24.3 mm² (+/−5.1) for saline-treated control wounds, heparanase-treated wounds area was 15.5 mm² (+/−3.1) (FIG. 1a), which represent a 40% decrease in wound area (FIG. 1b). Differences were found to be statistically significant (P=0.00238).

Microscopic Analysis of Heparanase Treated Wounds

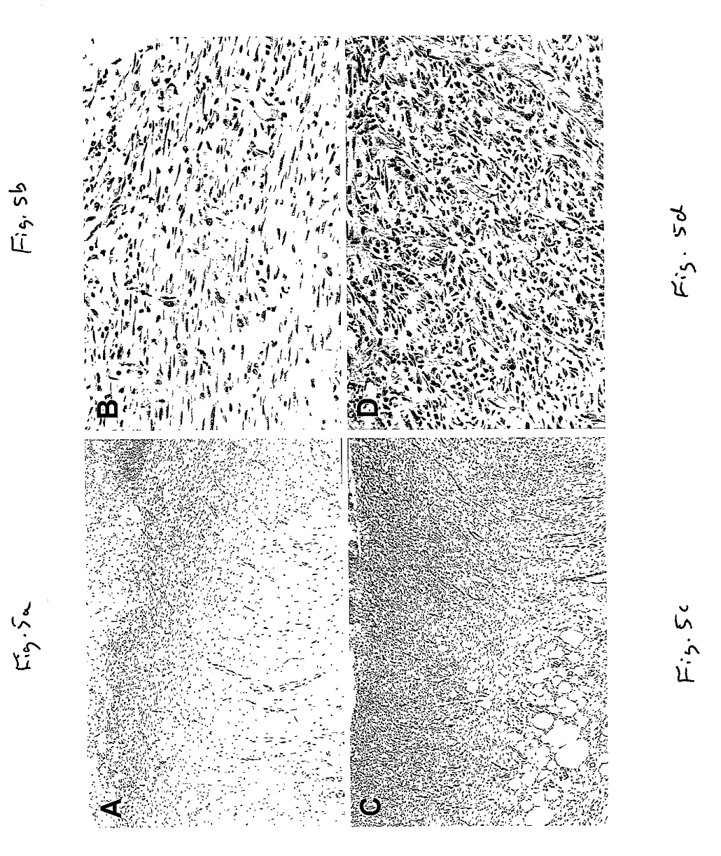

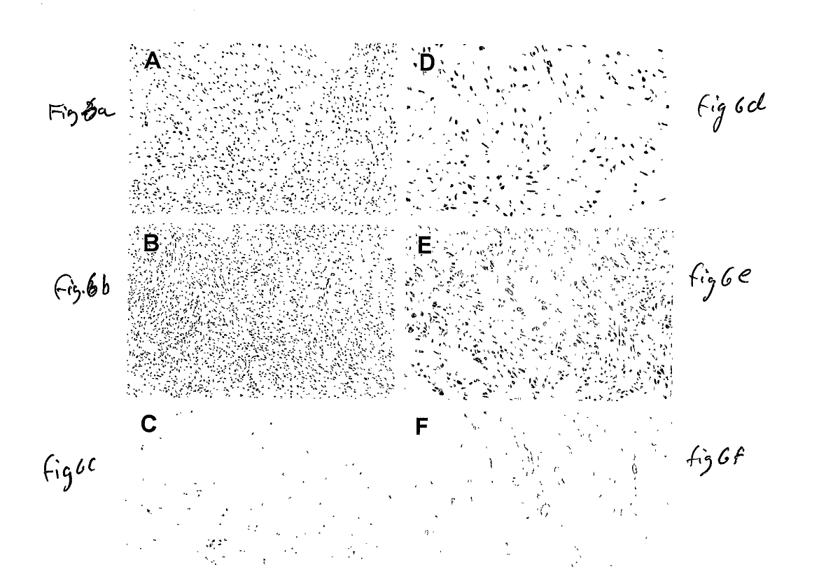

Having demonstrated, for the first time, a direct role for heparanase activity in the wound healing process, cellular and molecular mechanisms that are activated by heparanase in the course of wound healing were sought. Examination of hematoxilin-eosin stained wound sections revealed the expected granulation tissue morphology, composed of fibroblasts, blood vessels and inflammatory cells (FIGS. 5a-b). Interestingly, the heparanase-treated granulation tissue was much more dense. Specifically, a significant increase in the number of inflammatory cells and blood vessels was observed (FIGS. 5c-d). This was further confirmed by staining for PCNA, a marker for cell proliferation (FIGS. 6a-b and 6d-e) and for PECAM-1, a marker for endothelial cells (FIGS. 6c-f). Indeed, an increase in PCNA (FIGS. 6d-e) and PECAM-1 (FIGS. 6c and 6f) staining was observed in the granulation tissue of heparanase-treated wounds. Thus, the acceleration of wound healing may be due, without limitation, to the robust fibroblast and inflammatory cells-derived cytokine and chemokines and to increased vascularity.

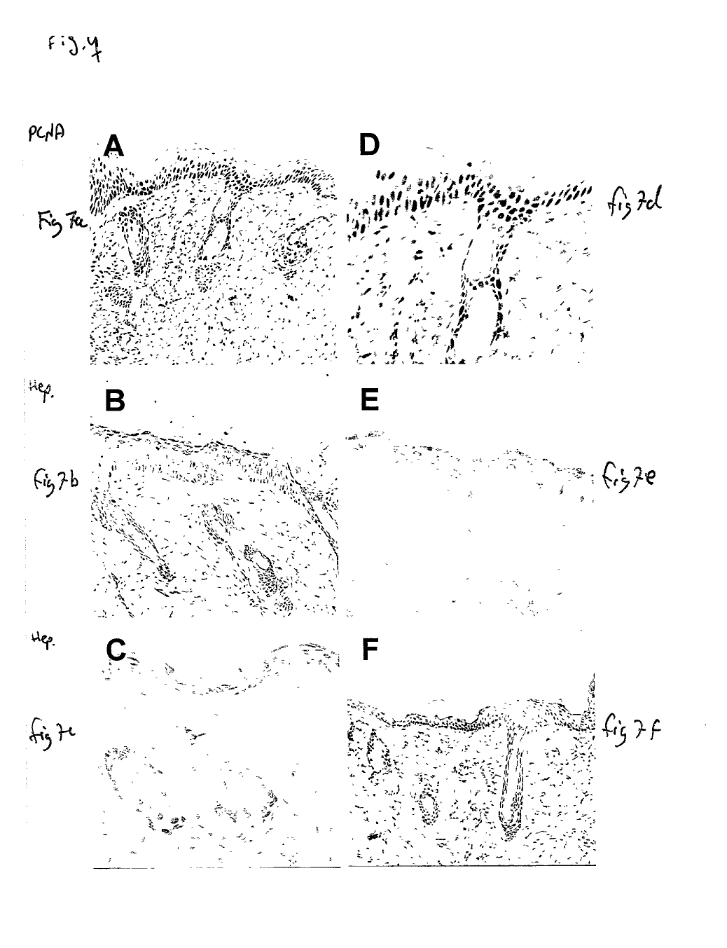

Heparanase was found to be expressed by all the major cell components of granulation tissue. Interestingly, heparanase expression was mainly detected in the differentiated, non-proliferating, cells composing the epidermis (FIGS. 7b and 7e-f), while proliferating, PCNA-positive epidermal cells (FIG. 7a and 7d) reconstituting the wound were poorly stained. In addition, heparanase staining was observed in non-proliferating sebaceous glands (compare FIGS. 7a and 7d with FIG. 7c) cells. Such staining pattern suggests, without limitation, that heparanase plays a role in cellular terminal differentiation which leads, as in the case of keratinocyes, to apoptosis and further as an anti-infectant.

Stimulation of Angiogenesis by Heparanase in Wounded Rat Eye Model

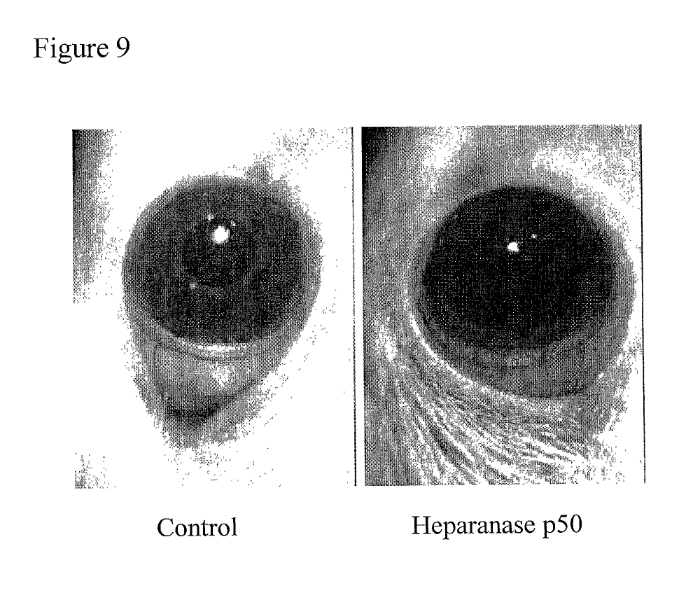

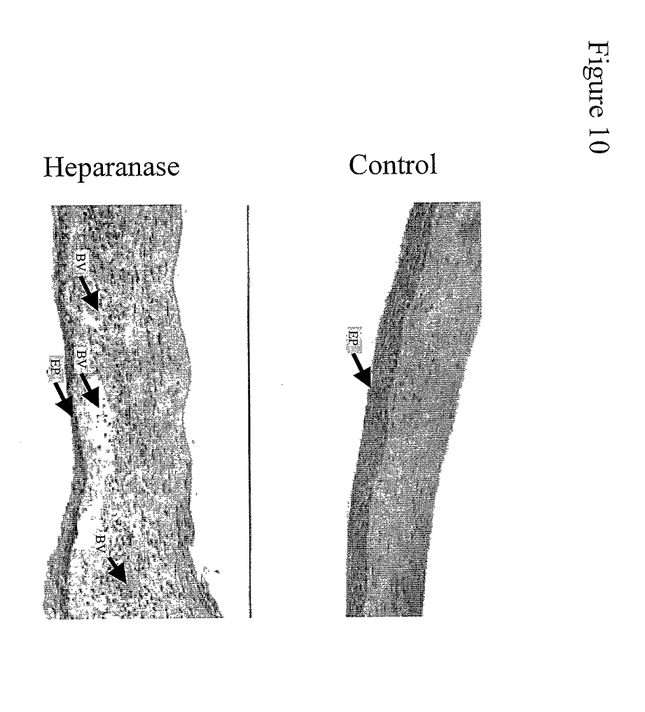

The central cornea of rats eyes was scraped with a surgical knife. The right eye of each rat was then treated with heparanase, 50 μl drop (1 mg/ml) of purified recombinant human P50 heparanase, three times a day. The left eye served as a control and was treated with Lyeteers. Vascularization and epithelialization were evaluated following closure of the corneal lesion. As shown in FIG. 9a heparanase treated eyes exhibited vascularization of the cornea, as well as increased vascularization in the iris. Normal, minor vascularization of the iris and non vascular appearance of the cornea were observed in the controls (FIG. 9). Histological examination of cornea from control eyes (FIG. 10) showed healing of the epithelia which is accompanied by a normal organized structure of the cornea while heparanase treatment (FIG. 10) resulted in growth of blood vessels into the cornea (arrows), followed by a massive infiltration of lymphocytes. Vascularization associated inflammatory reaction interfered with corneal healing, as demonstrated by a disorganized structure of the cornea.

Cosmetic Use

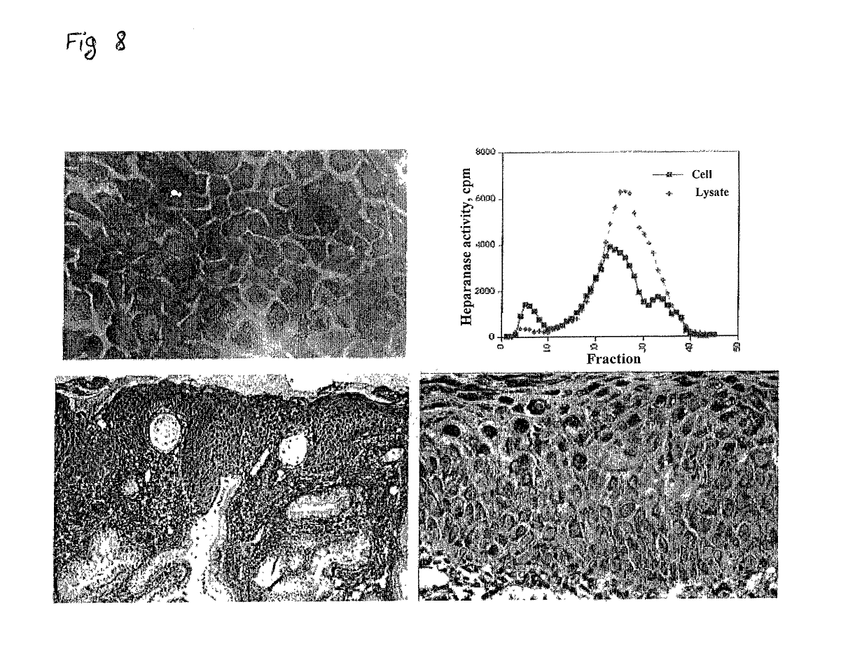

Using anti-heparanase monoclonal antibody (HP-92) cultures of HaCat keratinocytes cell line were immunostained. These cells exhibited significant heparanase staining in their cytoplasm (FIG. 8a). Moreover, intact cells, as well as an extract of these cells, exhibited heparanase activity when assayed in an ECM-assay (FIG. 8b). Immuno-staining of normal skin tissues resulted in the intense staining of heparanase both in the dermis and epidermis (FIGS. 8c-d).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method of inducing or accelerating a healing process of a wound, the method comprising the step of administering to the wound a therapeutically effective amount of heparanase, so as to induce or accelerate the healing process of the wound.

2. The method of claim 1, wherein said wound is selected from the group consisting of an ulcer, a burn, laceration, a surgical incision, necrosis and a pressure wound.

3. The method of claim 2, wherein said ulcer is a diabetic ulcer.

4. The method of claim 1, wherein said heparanase is recombinant.

5. The method of claim 1, wherein said heparanase is of a natural source.

6. The method of claim 1, wherein said heparanase is contained in a pharmaceutical composition adapted for topical application.

7. The method of claim 6, wherein said pharmaceutical composition is selected from the group consisting of an aqueous solution, a gel, a cream, a paste, a lotion, a spray, a suspension, a powder, a dispersion, a salve and an ointment.

8. The method of claim 6, wherein said pharmaceutical composition includes a solid support.

9. A method of inducing or accelerating a healing process of a wound, the method compromising the step of implanting into the wound a therapeutically effective amount of heparanase expressing or secreting cells, or heparanase coated cells, so as to induce or accelerate the healing process of the wound.

10. The method of claim 9, wherein said wound is selected from the group consisting of an ulcer, a burn, a laceration, a surgical incision, necrosis and a pressure wound.

11. The method of claim 10, wherein said ulcer is a diabetic ulcer.

12. The method of claim 9, wherein said cells are transformed to produce and secrete heparanase.

13. The method of claim 12, wherein said cells are transformed by a cis-acting element sequence integrated upstream to an endogenous heparanase gene of said cells and therefore said cells produce and secrete natural heparanase.

14. The method of claim 12, wherein said cells are transformed by a recombinant heparanase gene and therefore said cells produce and secrete recombinant heparanase.

15. The method of claim 9, wherein said heparanase expressing or secreting cells are capable of forming secretory granules.

16. The method of claim 9, wherein said heparanase expressing or secreting cells are endocrine cells.

17. The method of claim 9, wherein said heparanase expressing or secreting cells are of a human source.

18. The method of claim 9, wherein said heparanase expressing or secreting cells are of a histocompatibility humanized animal source.

19. The method of claim 9, wherein said heparanase expressing or secreting cells secrete human heparanase.

20. The method of claim 9, wherein said heparanase expressing or secreting cells are autologous cells.

21. The method of claim 9, wherein said cells are selected from the group consisting of fibroblasts, epithelial cells and keratinocytes.

22. A method of inducing or accelerating a healing process of a wound, the method compromising the step of transforming cells of the wound to produce and secrete heparanase, so as to induce or accelerate the healing process of the wound.

23. The method of claim 22, wherein said wound is selected from the group consisting of an ulcer, a burn, a laceration, a surgical incision, necrosis and a pressure wound.

24. The method of claim 23, wherein said ulcer is a diabetic ulcer.

25. The method of claim 22, wherein said cells are transformed by a cis-acting element sequence integrated upstream to an endogenous heparanase gene of said cells and therefore said cells produce and secrete natural heparanase.

26. The method of claim 22, wherein said cells are transformed by a recombinant heparanase gene and therefore said cells produce and secrete recombinant heparanase.

27. A pharmaceutical composition for inducing or accelerating a healing process of a wound, the pharmaceutical composition comprising, as an active ingredient, heparanase and a pharmaceutically acceptable carrier for topical application of the pharmaceutical composition.

28. The pharmaceutical composition of claim 27, packed and identified for treatment of wounds.

29. The pharmaceutical composition of claim 27, wherein said heparanase is recombinant.

30. The pharmaceutical composition of claim 27, wherein said heparanase is of a natural source.

31. The pharmaceutical composition of claim 27, wherein said pharmaceutical composition is selected from the group consisting of an aqueous solution, a gel, a cream, a paste, a lotion, a spray, a suspension, a powder, a dispersion, a salve and an ointment.

32. The pharmaceutical composition of claim 27, wherein said pharmaceutical composition includes a solid support.

33. A pharmaceutical composition for inducing or accelerating a healing process of a wound, the pharmaceutical composition comprising, as an active ingredient, heparanase expressing or secreting cells, or heparanase coated cells, and a pharmaceutically acceptable carrier being designed for topical application of the pharmaceutical composition.

34. The pharmaceutical composition of claim 33, packed and identified for treatment of wounds.

35. The pharmaceutical composition of claim 33, wherein said cells are transformed to produce and secrete heparanase.

36. The pharmaceutical composition of claim 33, wherein said cells are transformed by a cis-acting element sequence integrated upstream to an endogenous heparanase gene of said cells and therefore said cells produce and secrete natural heparanase.

37. The pharmaceutical composition of claim 33, wherein said cells are transformed by a recombinant heparanase gene and therefore said cells produce and secrete recombinant heparanase.

38. The pharmaceutical composition of claim 33, wherein said heparanase expressing or secreting cells are capable of forming secretory granules.

39. The pharmaceutical composition of claim 33, wherein said heparanase expressing or secreting cells are endocrine cells.

40. The pharmaceutical composition of claim 33, wherein said heparanase expressing or secreting cells are of a human source.

41. The pharmaceutical composition of claim 33, wherein said heparanase expressing or secreting cells are of a histocompatibility humanized animal source.

42. The pharmaceutical composition of claim 33, wherein said heparanase expressing or secreting cells secrete human heparanase.

43. The pharmaceutical composition of claim 33, wherein said heparanase expressing or secreting cells are autologous cells.

44. The pharmaceutical composition of claim 33, wherein said cells are selected from the group consisting of fibroblasts, epithelial cells, keratinocytes and cells present in a full thickness skin.

45. A pharmaceutical composition for inducing or accelerating a healing process of a wound, the pharmaceutical composition comprising, as an active ingredient, a nucleic acid construct being designed for transforming cells of said wound to produce and secrete heparanase, and a pharmaceutically acceptable carrier being designed for topical application of the pharmaceutical composition.

46. The pharmaceutical composition of claim 45, packed and identified for treatment of wounds.

47. The pharmaceutical composition of claim 45, wherein said cells are transformed by a cis-acting element sequence integrated upstream to an endogenous heparanase gene of said cells and therefore said cells produce and secrete natural heparanase.

48. The pharmaceutical composition of claim 45, wherein said cells are transformed by a recombinant heparanase gene and therefore said cells produce and secrete recombinant heparanase.

49. A method of inducing or accelerating angiogenesis, the method comprising the step of administering a therapeutically effective amount of heparanase, so as to induce or accelerate angiogenesis.

50. The method of claim 49, wherein said heparanase is recombinant.

51. The method of claim 49, wherein said heparanase is of a natural source.

52. The method of claim 49, wherein said heparanase is contained in a pharmaceutical composition.

53. The method of claim 52, wherein said pharmaceutical composition is selected from the group consisting of an aqueous solution, a gel, a cream, a paste, a lotion, a spray, a suspension, a powder, a dispersion, a salve and an ointment.

54. The method of claim 52, wherein. said pharmaceutical composition includes a solid support.

55. A method of inducing or accelerating angiogenesis, the method compromising the step of implanting a therapeutically effective amount of heparanase expressing or secreting cells, or heparanase coated cells, so as to induce or accelerate angiogenesis.

56. The method of claim 55, wherein said cells are transformed to produce and secrete heparanase.

57. The method of claim 56, wherein said cells are transformed by a cis-acting element sequence integrated upstream to an endogenous heparanase gene of said cells and therefore said cells produce and secrete natural heparanase.

58. The method of claim 56, wherein said cells are transformed by a recombinant heparanase gene and therefore said cells produce and secrete recombinant heparanase.

59. The method of claim 55, wherein said heparanase expressing or secreting cells are capable of forming secretory granules.

60. The method of claim 55, wherein said heparanase expressing or secreting cells are endocrine cells.

61. The method of claim 55, wherein said heparanase expressing or secreting cells are of a human source.

62. The method of claim 55, wherein said heparanase expressing or secreting cells are of a histocompatibility humanized animal source.

63. The method of claim 55, wherein said heparanase expressing or secreting cells secrete human heparanase.

64. The method of claim 55, wherein said heparanase expressing or secreting cells are autologous cells.

65. The method of claim 55, wherein said cells are selected from the group consisting of fibroblasts, epithelial cells, keratinocytes and cells present in a full thickness skin.

66. A method of inducing or accelerating angiogenesis, the method compromising the step of transforming cells in vivo to produce and secrete heparanase, so as to induce or accelerate angiogenesis.

67. The method of claim 66, wherein said cells are transformed by a cis-acting element sequence integrated upstream to an endogenous heparanase gene of said cells and therefore said cells produce and secrete natural heparanase.

68. The method of claim 66, wherein said cells are transformed by a recombinant heparanase gene and therefore said cells produce and secrete recombinant heparanase.

69. A pharmaceutical composition for inducing or accelerating angiogenesis, the pharmaceutical composition comprising, as an active ingredient, heparanase and a pharmaceutically acceptable carrier.

70. The pharmaceutical composition of claim 69, packed and identified for treatment of inducing or accelerating angiogenesis.

71. The pharmaceutical composition of claim 69, wherein said heparanase is recombinant.

72. The pharmaceutical composition of claim 69, wherein said heparanase is of a natural source.

73. The pharmaceutical composition of claim 69, wherein said pharmaceutical composition is selected from the group consisting of an aqueous solution, a gel, a cream, a paste, a lotion, a spray, a suspension, a powder, a dispersion, a salve and an ointment.

74. The pharmaceutical composition of claim 69, wherein said pharmaceutical composition includes a solid support.

75. A pharmaceutical composition for inducing or accelerating angiogenesis, the pharmaceutical composition comprising, as an active ingredient, heparanase expressing or secreting cells, or heparanase coated cells, and a pharmaceutically acceptable carrier.

76. The pharmaceutical composition of claim 75, packed and identified for inducing or accelerating angiogenesis.

77. The pharmaceutical composition of claim 75, wherein said cells are transformed to produce and secrete heparanase.

78. The pharmaceutical composition of claim 75, wherein said cells are transformed by a cis-acting element sequence integrated upstream to an endogenous heparanase gene of said cells and therefore said cells produce and secrete natural heparanase.

79. The pharmaceutical composition of claim 75, wherein said cells are transformed by a recombinant heparanase gene and therefore said cells produce and secrete recombinant heparanase.

80. The pharmaceutical composition of claim 75, wherein said heparanase expressing or secreting cells are capable of forming secretory granules.

81. The pharmaceutical composition of claim 75, wherein said heparanase expressing or secreting cells are endocrine cells.

82. The pharmaceutical composition of claim 75, wherein said heparanase expressing or secreting cells are of a human source.

83. The pharmaceutical composition of claim 75, wherein said heparanase expressing or secreting cells are of a histocompatibility humanized animal source.

84. The pharmaceutical composition of claim 75, wherein said heparanase expressing or secreting cells secrete human heparanase.

85. The pharmaceutical composition of claim 75, wherein said heparanase expressing or secreting cells are autologous cells.

86. The pharmaceutical composition of claim 75, wherein said cells are selected from the group consisting of fibroblasts, epithelial cells, keratinocytes and cells present in a full thickness skin.

87. A pharmaceutical composition for inducing or accelerating angiogenesis, the pharmaceutical composition comprising, as an active ingredient, a nucleic acid construct being designed for transforming cells in vivo to produce and secrete heparanase, and a pharmaceutically acceptable carrier.

88. The pharmaceutical composition of claim 87, packed and identified for inducing or accelerating angiogenesis.

89. The pharmaceutical composition of claim 87, wherein said cells are transformed by a cis-acting element sequence integrated upstream to an endogenous heparanase gene of said cells and therefore said cells produce and secrete natural heparanase.

90. The pharmaceutical composition of claim 87, wherein said cells are transformed by a recombinant heparanase gene and therefore said cells produce and secrete recombinant heparanase.
